(19) United States
(12) Patent Application Publication
(10) Pub. No.: US 2019/0286234 A1
(43) Pub. Date: Sep. 19, 2019

(54) SYSTEM AND METHOD FOR SYNCHRONIZED NEURAL MARKETING IN A VIRTUAL ENVIRONMENT

(71) Applicant: MindMaze Holdiing SA, Lausanne (CH)

(72) Inventor: Frederic CONDOLO, Lausanne (CH)

(21) Appl. No.: 16/357,410

(22) Filed: Mar. 19, 2019

Related U.S. Application Data
(60) Provisional application No. 62/644,732, filed on Mar. 19, 2018.

(51) Int. Cl. G06F 3/01; A61B 5/04; A61B 5/0476; A61B 5/00; A61B 5/0488; A61B 5/0496; A61B 5/0402; A61B 5/053; A61B 5/08; A61B 5/01; G06Q 30/02

(52) U.S. Cl. CPC G06F 3/015; H04N 5/91; A61B 5/0476; A61B 5/7285; A61B 5/0488; A61B 5/0496; A61B 5/0402; A61B 5/0533; A61B 5/0816; A61B 5/01; A61B 5/7425; G06Q 30/0242; G06F 3/012; A61B 2503/12; G06F 3/013; G06F 3/011; A61B 5/04012

(57) ABSTRACT

A system and method for determining a user reaction to images and/or sounds, for example in a video stream, for example as related to an advertisement. Optionally, the system and method are able to determine the user reaction to at least viewing and preferably handling a physical object, for example through an AR (augmented reality) headset.

### FIELD OF THE INVENTION

The present invention relates generally to a system to measure a physiological parameter of a user in response to a stimulus, and to provide feedback to the user.

### DESCRIPTION OF RELATED ART

Virtual reality-based systems have been used for various purposes, including gaming and the rehabilitation of patients who have suffered a stroke. For example, a VR-based system for rehabilitation of a patient is disclosed in “The design of a real-time, multimodal biofeedback system for stroke patient rehabilitation,” Chen, Y et al, ACM International Conference on Multimedia, 23 Oct. 2006 wherein infra-red cameras are used to track a 3-dimensional position of markers on an arm of a patient. Using a monitor, in VR a position of the arm of the patient is displayed as predefined movement patterns are completed, such as the grasping of a displayed image.

A drawback of certain VR-based systems is that they only measure the response of a body part to an instructed task or during an activity. Accordingly, they do not directly measure cortical activity in response to a displayed movement of a body part, only the way in which an area of the brain can control a body part, or other stimuli. This may lead to an inability to directly monitor a particular area of the brain. Moreover, the user is not fully immersed in the VR environment since they look to a separate monitor screen to view the VR environment.

One important drawback of known systems is that they do not reliably nor accurately control synchronization between stimulation or action signals and brain activity signals, which may lead to incorrect or inaccurate processing and read out of brain response signals as a function of stimuli or actions.

In conventional systems, in order to synchronize multimodal data (including physiological, behavioral, environmental, multimedia and haptic, among others) with stimulation sources (e.g., display, audio, electrical or magnetic stimulation) several independent, dedicated (i.e., for each data source) units are connected in a decentralized fashion, meaning that each unit brings its inherent properties (module latencies and jitters) into the system. Additionally, these units may have different clocks, therefore acquiring heterogeneous data with different formats and at different speeds. In particular, there is no comprehensive system that comprises stereoscopic display of virtual and/or augmented reality information, where some content may be related to some extent to the physiological/behavioral activity of any related user and registered by the system, and/or any information coming from the environment. Not fulfilling the above-mentioned requirements may have negative consequences in various cases in different application fields, as briefly mentioned in the following non-exhaustive list of examples:

a) Analysis of neural responses to stimulus presentation is of importance in many applied neuro-science fields. Current solutions compromise the synchronization quality, especially in the amount of jitter between the measured neural signal (e.g., EEG) and the simulation signal (e.g., display of a cue). Due to this, not only the signal to noise ratio of acquired signals is lowered but also limit the analysis to lower frequencies (typically less than 30 Hz). A better synchronization ensuring least jitter would open up new possibilities of neural signals exploration in the higher frequencies as well as precise (sub-millisecond) timing-based stimulation (not only non-invasive stimulation, but also invasive stimulation directly at the neural cite and subcutaneous stimulation).

b) Virtual reality and body perception: If the synchronization between the capture of user's movements and their mapping onto a virtual character (avatar) that reproduces the movement in real time is not achieved, then, the delayed visual feedback of the performed movement via a screen or head-mounted display will give to the user the feeling that he/she is not the author of such movement. This may have relatively important consequences in a number of contexts, including motor rehabilitation, where users are trained to recover mobility; training or execution of extremely dangerous operations such as deactivating a bomb by manipulating a robot remotely; game play where player immersion is important; commercial VR applications where potential-customer engagement through immersion is important; and the like.

c) Brain-computer interfaces: If the synchronization between motor intention (as registered by electroencephalographic data), muscle activity and the output towards a brain body-controlled neuroprosthesis fails, it is not possible to link motor actions with neural activation, preventing knowledge about the neural mechanisms underlying motor actions necessary to successfully control the neuroprosthesis.

d) Neurological examinations: The spectrum of electroencephalographic (EEG) data may reach up to 100 Hz for superficial, non-invasive recordings. In such a case, the time resolution is in the range of tens of milliseconds. If the synchronization between EEG and events evoking specific brain responses (e.g., P300 response for a determined action happening in virtual environments) fails, then it is not possible to relate the brain response to the particular event that elicited it.

### SUMMARY OF THE INVENTION

According to at least some embodiments of the present invention, there is provided a system and method for measuring a physiological parameter of a user to monitor cortical activity in response to a displayed movement of a body part, wherein the displayed movement is displayed to the user in a virtual or augmented reality. The system may be used to treat/aid recovery from neurological injury and/or neurological disease of the user after the user experiences a stroke. However, the system may be used in other applications such as gaming or learning of motor skills that may be required for a sports-related or other activity.

According to at least some embodiments, there is provided a system and method for determining a user reaction to images and/or sounds, for example in a video stream, for example as related to an advertisement. Optionally, the system and method are able to determine the user reaction to at least viewing and preferably handling a physical object, for example through an AR (augmented reality) headset.

Preferably the physiological parameter measurement and motion tracking system (e g, movements head and body) ensures accurate real time integration of measurement and control of physiological stimuli and response signals.

Optionally the physiological parameter measurement and motion tracking system can generate a plurality of stimuli signals of different sources (e.g., visual, auditive, touch sensory, electric, magnetic) and/or that can measure a plurality of physiological response signals of different types (e.g., brain activity, body part movement, eye movement, galvanic skin response).

According to at least some embodiments, the system is configured to reduce electrical interference among the input modules (measurements) and output modules (stimuli) and system operation.

According to at least some embodiments of the present invention, there is provided a system that is portable and simple to use such that it may be adapted for home use, for ambulatory applications, or for mobile applications. The system is preferably configured to be easily adapted to various head and body sizes, which is comfortable to wear, and which can be easily attached and removed from a user.

According to at least some embodiments of the present invention, there is provided a system that includes an optimized amount of brain activity sensors that provide sufficient brain activity yet save time for placement and operation. It would be advantageous to have different electrode configurations to easily adapt to target brain areas as required.

Preferably the system allows removal of a head mounted display without disturbing brain activity and other physiological and motion tracking modules to allow a pause for user.

Preferably the system has the ability to switch the display between AR and VR for see-through effect whenever needed without removing the HMD.

According to at least some embodiments of the present invention, there is provided a physiological parameter measurement and motion tracking system comprising a control system, a sensing system, and a stimulation system, the sensing system comprising one or more physiological sensors including at least brain electrical activity sensors, the stimulation system comprising one or more stimulation devices including at least a visual stimulation system, the control system comprising an acquisition module configured to receive sensor signals from the sensing system, and a control module configured to process the signals from the acquisition module and control the generation of stimulation signals to one or more devices of the stimulation system. The control system further comprises a clock module, wherein the control system is configured to receive signals from the stimulation system and to time stamp the stimulation system signals and the sensor signals with a clock signal from the clock module. The stimulation system signals may be content code signals transmitted from the stimulation system.

Brain activity sensors may include contact (EEG) or non contact sensors (MRI, PET), invasive (single- and multi-electrode arrays) and non invasive (EEG, MEG) sensors for brain monitoring.

The sensing system may further comprise a physiological sensor including any one or more of an Electromyogram (EMG) sensor, an Electrooculography (EOG) sensor, an Electrocardiogram (ECG) sensor, an inertial sensor, a body temperature sensor, and a galvanic skin sensor, respiration sensor, pulse oximetry.

The sensing system may further comprise position and/or motion sensors to determine the position and/or the movement of a body part of the user.

In an embodiment, at least one position/motion sensor comprises a camera and optionally a depth sensor.

The stimulation system may further comprise stimulation devices including any one or more of an audio stimulation device (33), a Functional Electrical Stimulation (FES) device (31), robotic actuator and a haptic feedback device.

According to at least some embodiments of the present invention, there is provided a physiological parameter measurement and motion tracking system comprising: a display system to display information to a user; a physiological parameter sensing system comprising one or more sensing means configured to sense electrical activity in a brain of a user and to generate brain electrical activity information; a position/motion detection system configured to provide a body part position information corresponding to a position/motion of a body part of the user; a control system arranged to receive the brain electrical activity information from the physiological parameter sensing system and to receive the body part position information from the position/motion detection system, the control system being configured to provide a target location information to the display system comprising a target location for the body part, the display system being configured to display the target location information, the control system being further configured to provide body part position information to the display system providing the user with a view of the movement of the body part, or an intended movement of the body part. The physiological parameter measurement and motion tracking system further comprises a clock module, the clock module being operable to time stamp information transferred from the physiological parameter sensing system and the position/motion detection system, the system being operable to process the information to enable real-time operation.

In an embodiment, the control system may be configured to determine whether there is no or an amount of movement less than a predetermined amount sensed by the position/motion detection system and if no or an amount of movement less than the predetermined amount is determined, then to provide the body part position information to the display system based at least partially on the brain electrical activity information, such that the displayed motion of the body part is at least partially based on the brain electrical activity information.

In an embodiment, the physiological parameter sensing system comprises a plurality of sensors configured to measure different physiological parameters, selected from a group including EEG sensor, ECOG sensor, EMG sensor, GSR sensor, respiration sensor, ECG sensor, temperature sensor, respiration sensor and pulse-oximetry sensor.

In an embodiment, the position/motion detection system comprises one or more cameras operable to provide an image stream of a user.

In an embodiment, the position/motion detection system comprises one or more cameras operable to provide an image stream of one or more objects in the scene.

In an embodiment, the position/motion detection system comprises one or more cameras operable to provide an image stream of one or more persons in the scene.

In an embodiment, the cameras comprise one or more color cameras and a depth sensing camera.

In an embodiment, the control system is operable to supply information to the physiological parameter sensing system cause a signal to be provided to stimulate movement or a state of a user.

In an embodiment, the system may further comprise a head set forming a single unit incorporating said display system operable to display a virtual or augmented reality image or video to the user; and said sensing means configured to sense electrical activity in a brain, the sensing means comprising a plurality of sensors distributed over a sensory and motor region of the brain of the user.

In an embodiment, the brain activity sensors are arranged in groups to measure electrical activity in specific regions of the brain.

In an embodiment, the display unit is mounted to a display unit support configured to extend around the eyes of a user and at least partially around the back of the head of the user.

In an embodiment, sensors are connected to a flexible cranial sensor support that is configured to extend over a head of a user. The cranial sensor support may comprise a plate and/or cap on which the sensors are mounted, the plate being connected to or integrally formed with a strap which is configured to extend around a top of a head of a user, the strap being connected at its ends to the display system support. The head set may thus form an easily wearable unit.

In an embodiment, the cranial sensor support may comprise a plurality of pads, a first group of pads being arranged to extend from a first pad support which extends in an approximately orthogonal direction from the display unit support, a second group of pads being arranged to extend from a second pad support which extends in an approximately orthogonal direction from the display unit support.

In an embodiment, the headset may incorporate a plurality of sensors configured to measure different physiological parameters, selected from a group comprising EEG sensors, an ECOG sensor, an eye movement sensor, and a head movement sensor.

In an embodiment, the headset may further incorporate one of said position/motion detection system operable to detect a position/motion of a body part of a user.

In an embodiment, the position/motion detection system may comprise one or more color cameras, and a depth sensor.

In an embodiment, the headset comprises a wireless data transmitting means configured to wirelessly transmit data from one or more of the following systems: the physiological parameter sensing system; the position/motion detection system; the head movement sensing unit.

In an embodiment, the system may further comprise a functional electrical stimulation (FES) system connect to the control system and operable to electrically stimulate one or more body parts of the user, the FES including one or more stimulation devices selected from a group consisting of electrodes configured to stimulate nerves or muscles, trans-cranial alternating current stimulation (tACS), direct current stimulation (tDCS), trans-cranial magnetic stimulation (TMS) and trans-cranial Ultrasonic stimulation.

In an embodiment, the system may further comprise a robotic system for driving movements of a limb of the user and configured to provide haptic feedback.

In an embodiment, the system may further comprise an exercise logic unit configured to generate visual display frames including instructions and challenges to the display unit.

In an embodiment, the system may further comprise an events manager unit configured to generate and transmit stimulation parameters to the stimulation unit.

In an embodiment, each stimulation device may comprise an embedded sensor whose signal is registered by a synchronization device.

In an embodiment, the system may further comprise a display register configured to receive display content representing a final stage before the display content is activated on the display, the display register being configured to generate a display content code for transmission to the control system, a time stamp being attached to the display content code by the clock module.

In an embodiment, the stimulation system comprises stimulation devices that may comprise audio stimulation device, Functional Electrical Stimulation (FES) devices, and haptic feedback devices.

The clock module may be configured to be synchronized with clock module of other systems, including external computers.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the apparatuses, devices, methods, and systems of the present disclosure involve performing or completing certain selected tasks or steps manually, automatically, or a combination thereof. Specifically, several selected steps can be implemented by hardware or by software on an operating system, of a firmware, and/or a combination thereof. For example, as hardware, selected steps of at least some embodiments of the disclosure can be implemented as a chip or circuit (e.g., ASIC). As software, selected steps of at least some embodiments of the disclosure can be implemented as a number of software instructions being executed by a computer (e.g., a processor of the computer) using an operating system. In any case, selected steps of methods of at least some embodiments of the disclosure can be described as being performed by a processor, such as a computing platform for executing a plurality of instructions.

Software (e.g., an application, computer instructions) which is configured to perform (or cause to be performed) certain functionality may also be referred to as a “module” for performing that functionality, and also may be referred to a “processor” for performing such functionality. Thus, processor, according to some embodiments, may be a hardware component, or, according to some embodiments, a software component.

Further to this end, in some embodiments: a processor may also be referred to as a module; in some embodiments, a processor may comprise one more modules; in some embodiments, a module may comprise computer instructions—which can be a set of instructions, an application, software—which are operable on a computational device (e.g., a processor) to cause the computational device to conduct and/or achieve one or more specific functionality. Furthermore, the phrase “abstraction layer” or “abstraction interface,” as used with some embodiments, can refer to computer instructions (which can be a set of instructions, an application, software) which are operable on a computational device (as noted, e.g., a processor) to cause the computational device to conduct and/or achieve one or more specific functionality. The abstraction layer may also be a circuit (e.g., an ASIC) to conduct and/or achieve one or more specific functionality. Thus, for some embodiments, and claims which correspond to such embodiments, the noted feature/functionality can be described/claimed in a number of ways (e.g., abstraction layer, computational device, processor, module, software, application, computer instructions, and the like).

Some embodiments are described with regard to a “computer”, a “computer network,” and/or a “computer operational on a computer network,” it is noted that any device featuring a processor (which may be referred to as “data processor”; “pre-processor” may also be referred to as “processor”) and the ability to execute one or more instructions may be described as a computer, a computational device, and a processor (e.g., see above), including but not limited to a personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, a pager, and/or a similar device. Two or more of such devices in communication with each other may be a “computer network.”

### BRIEF DESCRIPTION OF THE DRAWINGS

For a better understanding of the invention, and to show how embodiments of the same may be carried into effect, reference will now be made, by way of example, to the accompanying diagrammatic drawings in which:

FIGS. 1a and 1b are schematic illustrations of prior art systems;

FIG. 2a is a schematic diagram illustrating an embodiment of the invention in which display content displayed to a user is synchronized with response signals (e.g., brain activity signals) measured from the user;

FIG. 2b is a schematic diagram illustrating an embodiment of the invention in which audio content played to a user is synchronized with response signals (e.g., brain activity signals) measured from the user;

FIG. 2c is a schematic diagram illustrating an embodiment of the invention in which a plurality of signals applied to a user are synchronized with response signals (e.g., brain activity signals) measured from the user;

FIG. 2d is a schematic diagram illustrating an embodiment of the invention in which a haptic feedback system is included;

FIG. 2e is a schematic diagram illustrating an embodiment of the invention in which a neuro-stimulation signal is applied to a user;

FIG. 3a is a simplified schematic diagram of a physiological parameter measurement and motion tracking system according to the invention;

FIG. 3b is a detailed schematic diagram of a control system of the system of FIG. 3a;

FIG. 3c is a detailed schematic diagram of a physiological tracking module of the control system of FIG. 3b;

FIGS. 4a and 4b are perspective views of a headset according to an embodiment of the invention;

FIG. 5 is a plan view of an exemplary arrangement of EEG sensors on a head of a user;

FIG. 6 is a front view of an exemplary arrangement of EMG sensors on a body of a user;

FIG. 7 is a diagrammatic view of a process for training a stroke victim using an embodiment of the system;

FIGS. 8a-8g is a view of screen shots which are displayed to a user during the process of FIG. 7;

FIG. 9 is a perspective view of a physical setup of a physiological parameter measurement and feedback system according to an exemplary embodiment of the invention;

FIG. 10 is a schematic block diagram of an example stimulus and feedback trial of a physiological parameter measurement and feedback system according to an exemplary embodiment of the invention;

FIG. 11 is a schematic block diagram of an acquisition module of a physiological parameter measurement and feedback system according to an exemplary embodiment of the invention;

FIG. 12 is a diagram illustrating time stamping of a signal by a clock module of a physiological parameter measurement and feedback system according to an exemplary embodiment of the invention;

FIG. 13 is a data-flow diagram illustrating a method of processing physiological signal data in a control system of a physiological parameter measurement and feedback system according to an exemplary embodiment of the invention;

FIG. 14 is a flowchart diagram illustrating a method of processing events in a control system of a physiological parameter measurement and feedback system according to an exemplary embodiment of the invention;

FIG. 15a shows an exemplary, non-limiting schematic block diagram for measuring an effect of visual stimuli on a reaction of an individual in a virtual reality environment;

FIG. 15b shows an exemplary, non-limiting process for determining an effect of an advertisement on a user in a virtual reality environment;

FIG. 16a shows an exemplary, non-limiting schematic block diagram for measuring an effect of visual stimuli on a reaction of an individual in an augmented reality environment; and

FIG. 16b shows an exemplary, non-limiting process for determining an effect of an advertisement on a user in an augmented reality environment.

### DETAILED DESCRIPTION OF EXEMPLARY EMBODIMENTS

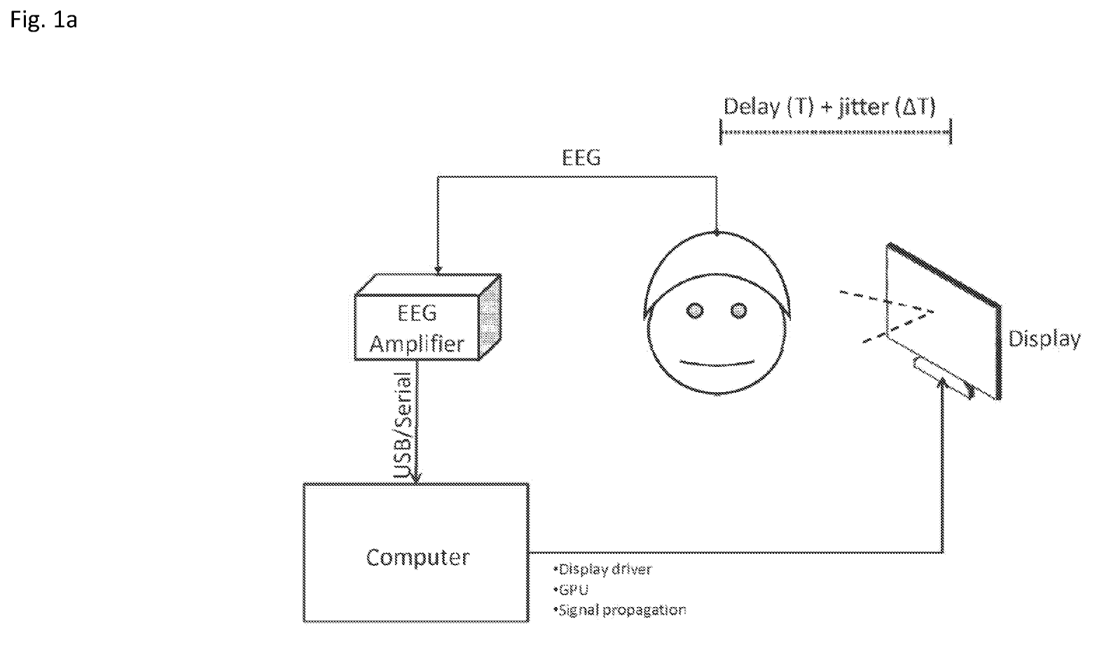

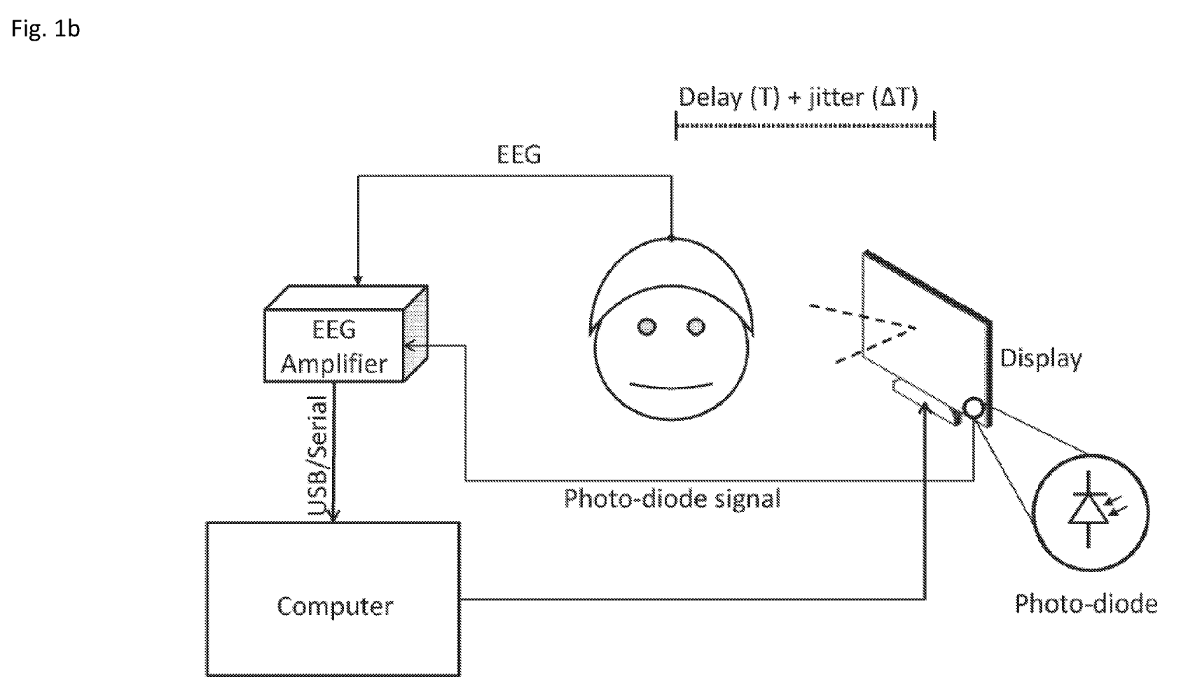

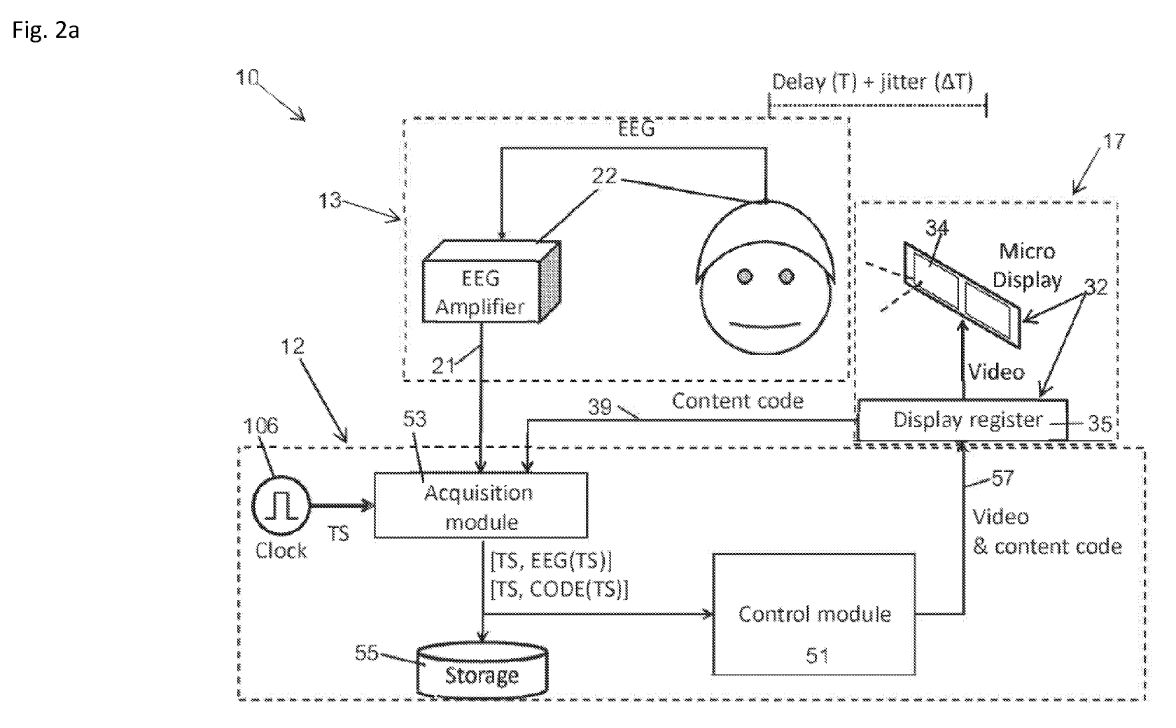

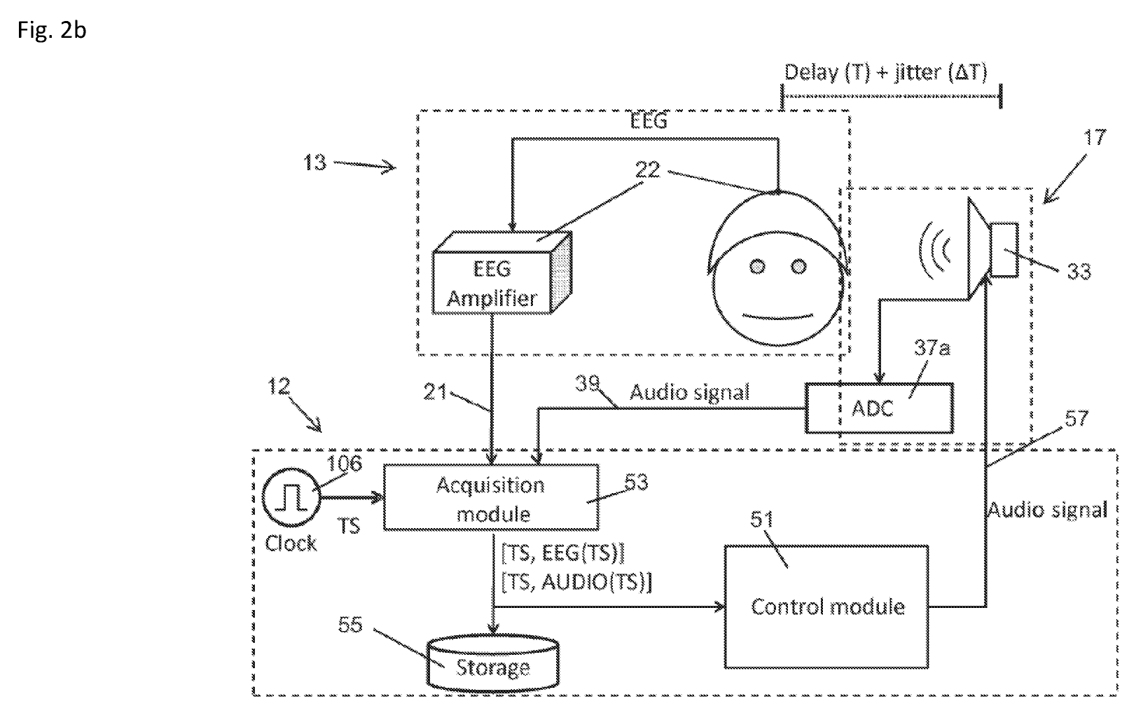

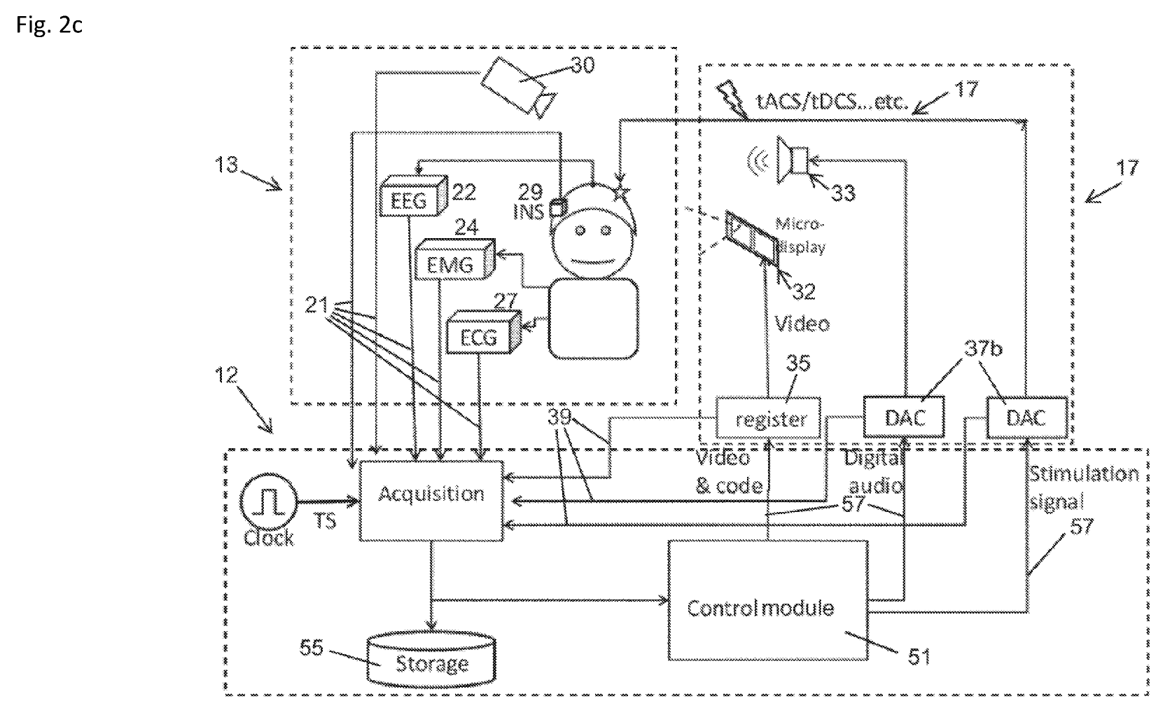

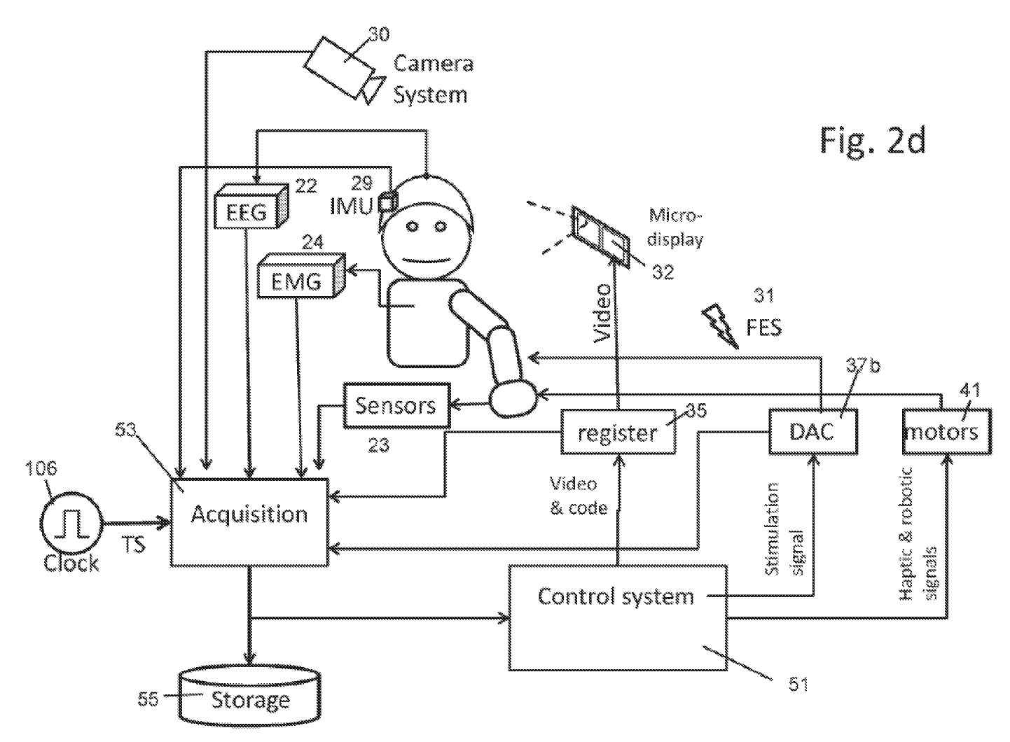

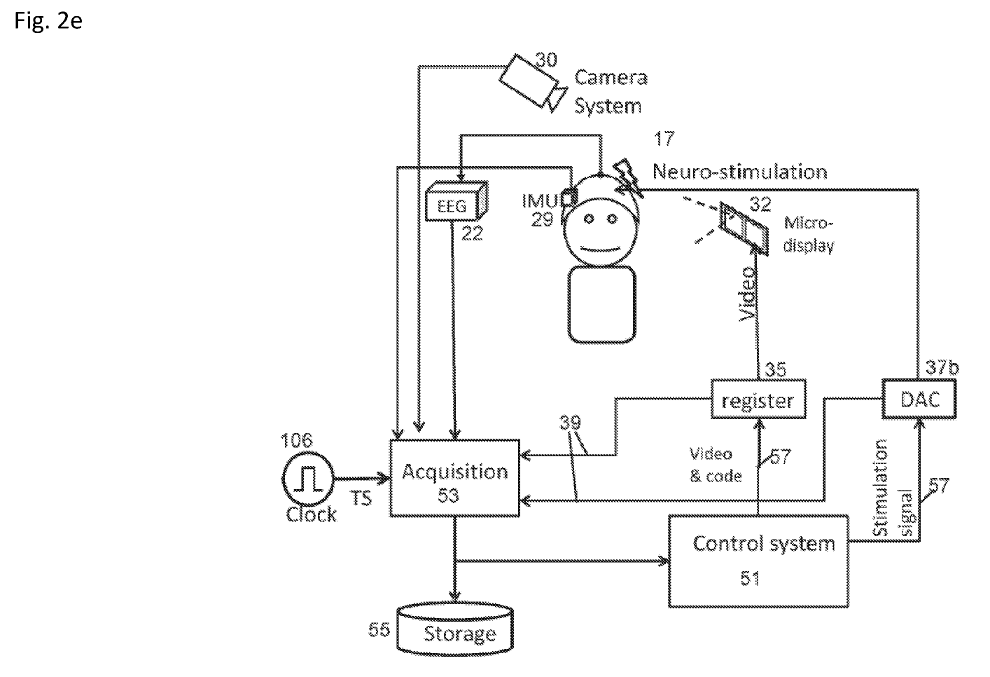

FIGS. 1a and 1b show conventional systems and are described in greater detail below. A physiological parameter measurement and motion tracking system according to embodiments of the invention is shown in FIGS. 2a-2e. FIG. 2a shows a system 10, featuring a control system 12, a sensing system 13, and a stimulation system 17. System 10 features synchronization between the content fed to a micro-display on the headset and brain activity signals (e.g., EEG signals), as schematically illustrated.

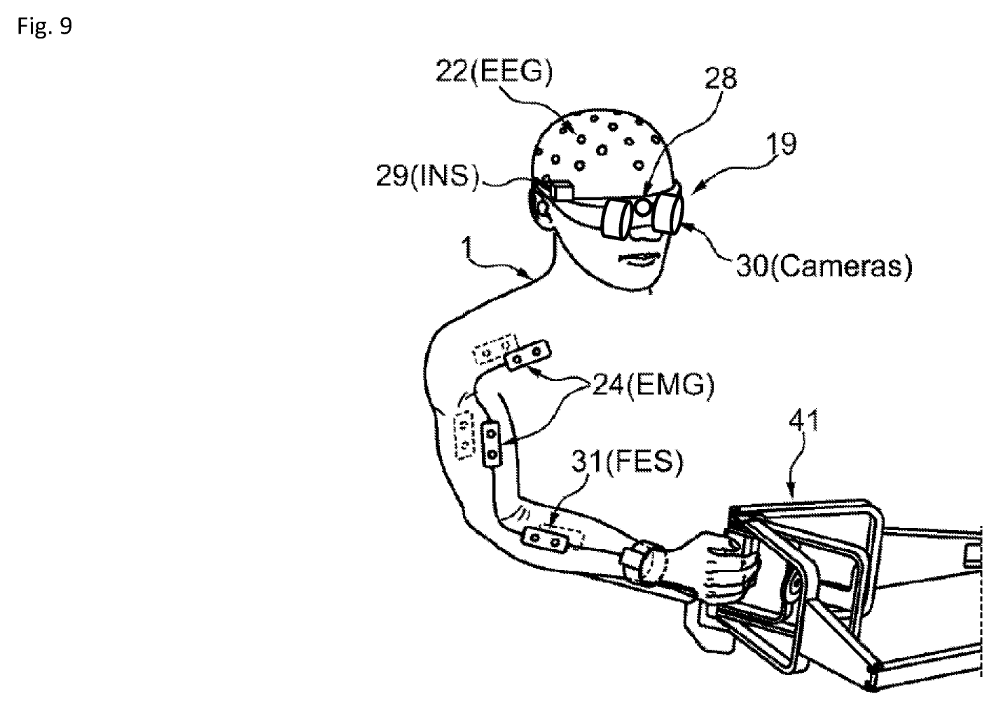

The sensing system 10 comprises one or more physiological sensors including at least brain electrical activity sensors, for instance in the form of electroencephalogram (EEG) sensors 22. The sensing system may comprise other physiological sensors selected from a group comprising electromyogram (EMG) sensors 24 connected to muscles in a user's body, electrooculography (EOG) sensors 25 (eye movement sensors), electrocardiogram (ECG) sensors 27, inertial sensors (INS) 29 mounted on the user's head and optionally on other body parts such as the user's limbs, body temperature sensor, and a galvanic skin sensor. The sensing system further comprises position and/or motion sensors to determine the position and/or the movement of a body part of the user. Position and motion sensors may further be configured to measure the position and/or movement of an object in the field of vision of the user. It may be noted that the notion of position and motion is related to the extent that motion can be determined from a change in position. In embodiments of the invention, position sensors may be used to determine both position and motion of an object or body part; or a motion sensor (such as an inertial sensor) may be used to measure movement of a body part or object without necessarily computing the position thereof. In an advantageous embodiment, at least one position/motion sensor comprises a camera 30 and optionally a distance sensor 28, mounted on a head set 18 (for example, as illustrated in FIG. 9) configured to be worn by the user.

The stimulation system 17 comprises one or more stimulation devices including at least a visual stimulation system 32. The stimulation system may comprise other stimulation devices selected from a group comprising audio stimulation device 33, and functional electrical stimulation (FES) devices 31 connected to the user (for instance to stimulate nerves, or muscles, or parts of the user's brain e.g., to stimulate movement of a limb), and haptic feedback devices (for instance a robot arm that a user can grasp with his hand and that provides the user with haptic feedback). The stimulation system may further comprise Analogue to Digital Converters (ADC) 37a and Digital to Analogue Converters (DAC) 37b for transfer and processing of signals by a control module 51 of the control system. Devices of the stimulation system may further advantageously comprise means to generate content code signals 39 fed back to the control system 12 in order to timestamp said content code signals and to synchronize the stimulation signals with the measurement signals generated by the sensors of the sensing system.

The control system 12 comprises a clock module 106 and an acquisition module 53 configured to receive content code signals from the stimulation system and sensor signals from the sensing system and to time stamp these signals with a clock signal from the clock module 106. The control system 12 further comprises a control module 51 that processes the signals from the acquisition module and controls the output of the stimulation signals to devices of the stimulation system 17. The control module 51 further comprises a memory 55 to store measurement results, control parameters and other information useful for operation of the physiological parameter measurement and motion tracking system 10.

Generally, the visual/video content that is generated in the control system 12 is first pushed to a display register 35 (a final stage before the video content is activated on the display). In our design together with video content, the controller sends a code to a part of the register (say N bits) corresponding to one or more pixels (not too many pixels, so that the user is not disturbed; the corner pixels in the micro display are recommended as they may not be visible to user). The code will be defined by controller describing what exactly is the display content. Now using a clock signal the acquisition module 53 reads the code from the display register 35 and attaches a time stamp and sends to next modules. At the same moment EEG samples are also sampled and attached with the same time stamp. This way when EEG samples and the video code samples are arrived at the controller, these samples could be interpreted accordingly.

Note that all these modules are employed in one embedded system that has a single clock. This leads to the least latency as well as least jitter.

The same principle may be used for an audio stimulation as illustrated in FIG. 2b. The audio stimulation can be sampled by the data sent to a digital to analog (DAC) converter.

More generally, any kind of stimulation, as illustrated in FIG. 2c, (such as trans-cranial stimulations (tACS), tDCS, TMS, etc.) could be directed to the acquisition module 53 using a sensor and an analog to digital (ADC) converter. This can also be achieved by sending the digital signals supplied to DAC as illustrated in the case of audio stimulation. Plural data from an EEG, video camera data or any other sensor (e.g., INS) is synchronized in the same framework. Note that each sensor or stimulation could be sampled with a different sampling frequency. The system is configured so that the sensor or stimulation data samples are attached with the time-stamp defined with the clock module.

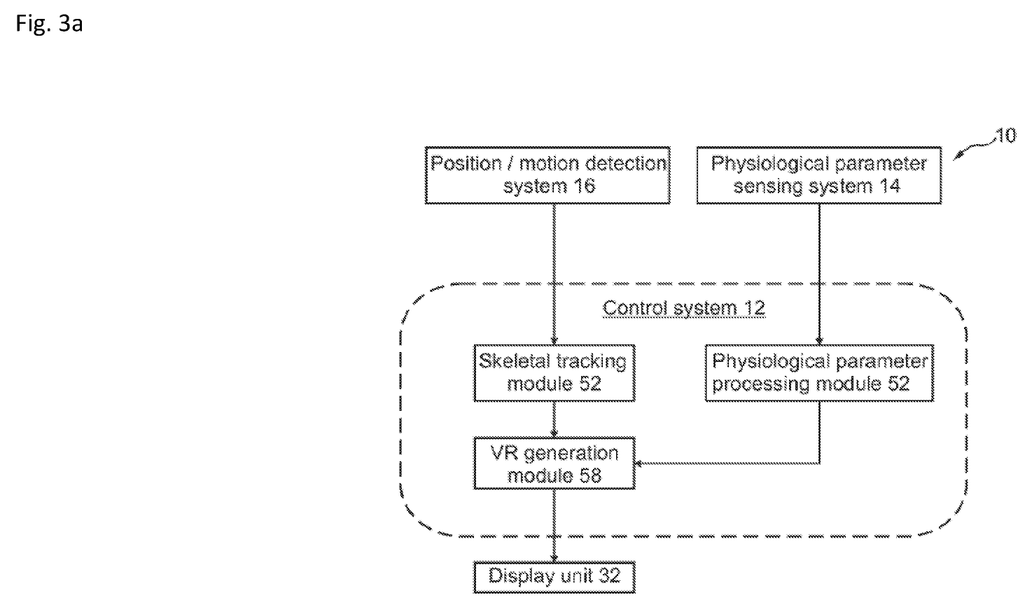

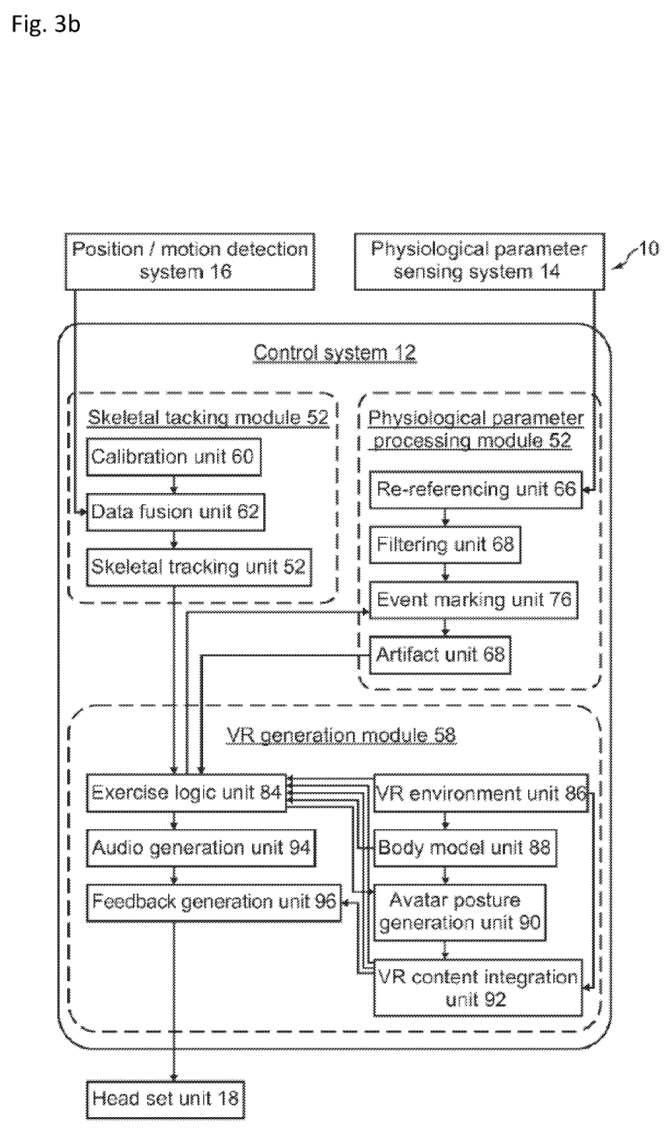

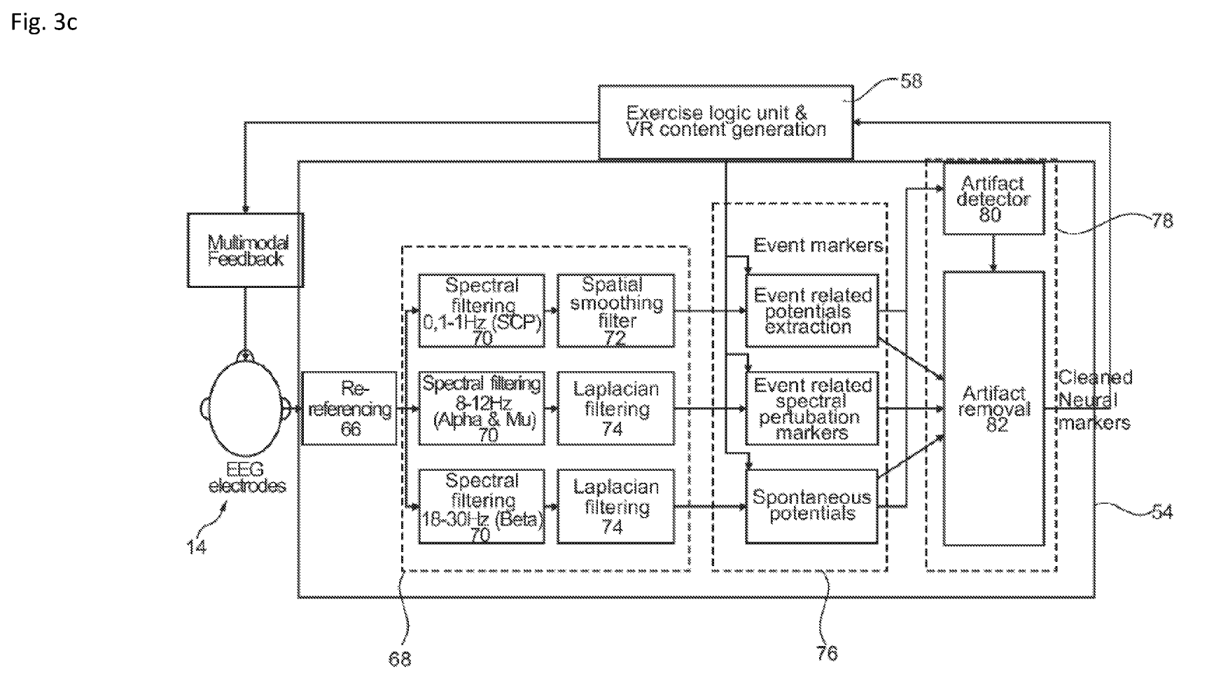

FIG. 3a is a simplified schematic diagram of a physiological parameter measurement and motion tracking system 10 according to an embodiment of the invention. The system 10 comprises a control system 12 which may be connected to one or more of the following units: a physiological parameter sensing system 14; position/motion detection system 16; and a head set 18, all of which will be described in more detail in the following.

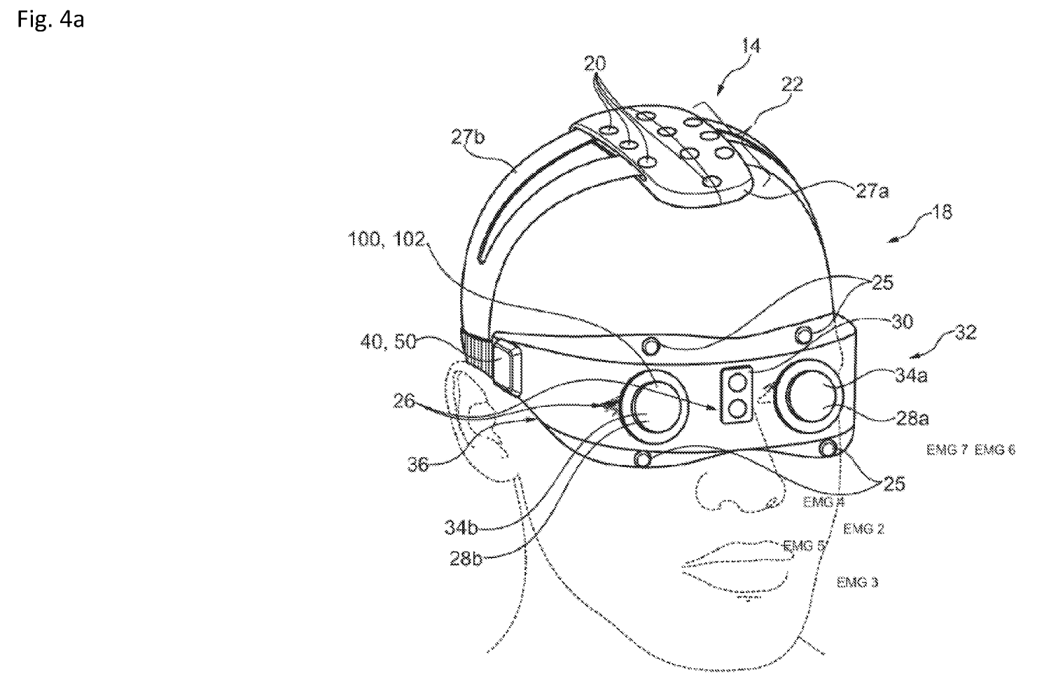

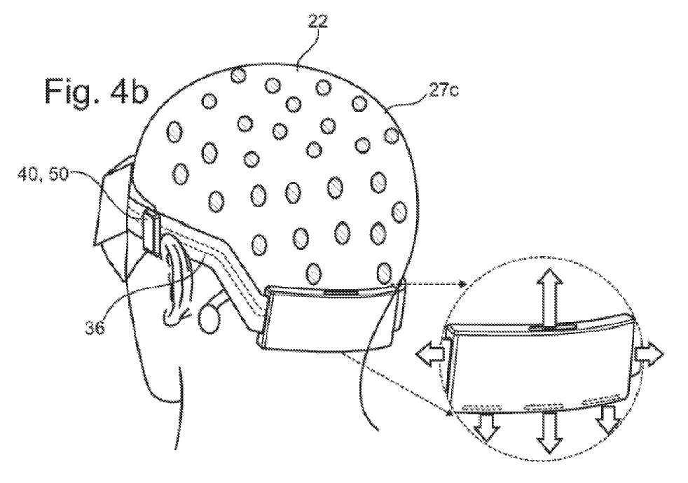

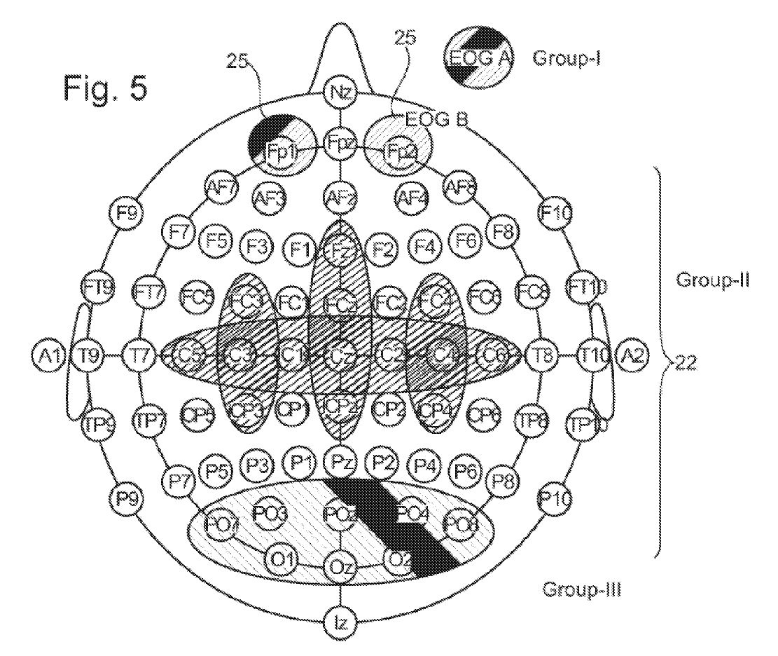

The physiological parameter sensing system 14 comprises one or more sensors 20 configured to measure a physiological parameter of a user. In an advantageous embodiment the sensors 20 comprise one or more sensors configured to measure cortical activity of a user, for example, by directly measuring the electrical activity in a brain of a user. A suitable sensor is an electroencephalogram (EEG) sensor 22. EEG sensors measure electrical activity along the scalp, such voltage fluctuations result from ionic current flows within the neurons of the brain. An example of suitable EEG sensors is a g.tec Medical Engineering GmbH g.scarabeo. FIG. 4a shows an exemplary arrangement of electroencephalogram sensors 22 on a head of a user. In this example, arrangement the sensors are arranged in a first group 22a such that cortical activity proximate a top of the head of the user is measured. FIG. 5 shows a plan view of a further exemplary arrangement, wherein the sensors are arranged into a first group 22c, second group 22d, and third group 22e. Within each group there may be further subsets of groups. The groups are configured and arranged to measure cortical activity in specific regions. The functionality of the various groups that may be included is discussed in more detail in the following. It will be appreciated that the present invention extends to any suitable sensor configuration.

In an advantageous embodiment, the sensors 22 are attached to a flexible cranial sensor support 27 which is made out of a polymeric material or other suitable material. The cranial sensor support 27 may comprise a plate 27a which is connected to a mounting strap 27b that extends around the head of the user, as shown in FIG. 4a. In another embodiment as shown in FIG. 4b, the cranial sensor support 27 may comprise a cap 27c, similar to a bathing cap, which extends over a substantial portion of a head of a user. The sensors are suitably attached to the cranial sensor support. For example, they may be fixed to or embedded within the cranial sensor support 27. Advantageously, the sensors can be arranged with respect to the cranial sensor support such that when the cranial sensor support is positioned on a head of a user the sensors 20 are conveniently arranged to measure cortical activity specific areas, for example those defined by the groups 22a, 22c-d in FIGS. 4 and 5. Moreover, the sensors 20 are conveniently fixed to and removed from the user.

In an advantageous embodiment, the size and/or arrangement of the cranial sensor support is adjustable to accommodate users with different head sizes. For example, the strap 27b may have adjustable portions or the cap may have adjustable portions in a configuration such as and adjustable strap found on a baseball cap.

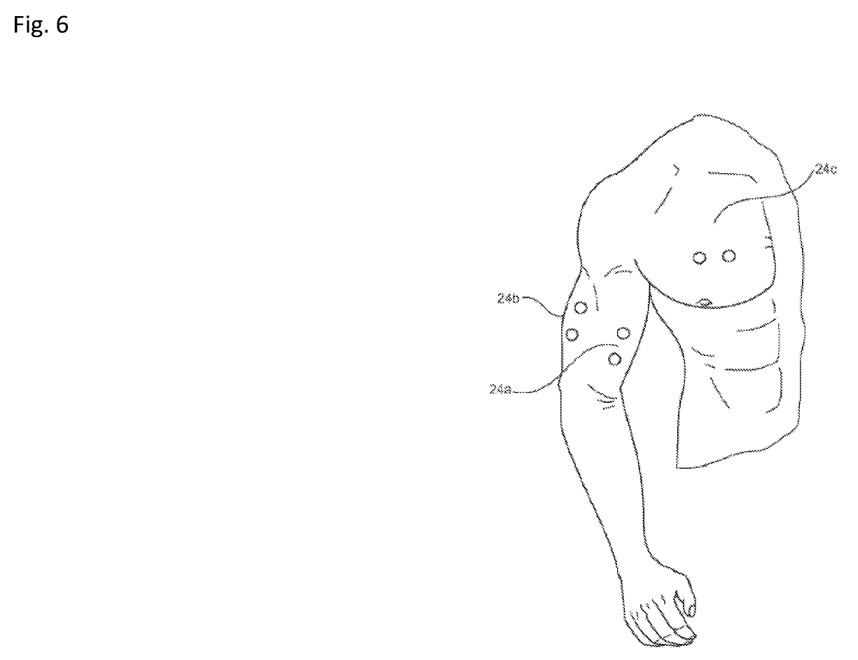

In an advantageous embodiment, one or more sensors 20 may additionally or alternatively comprise sensors 24 configured to measure movement of a muscle of a user, for example by measuring electrical potential generated by muscle cells when the cells are electrically or neurologically activated. A suitable sensor is an electromyogram EMG sensor. The sensors 24 may be mounted on various parts of a body of a user to capture a particular muscular action. For example, for a reaching task, they may be arranged on one or more of the hand, arm and chest. FIG. 6 shows an exemplary sensor arrangement, wherein the sensors 24 are arranged on the body in: a first group 24a on the biceps muscle; a second group 24b on the triceps muscle; and a third group 24c on the pectoral muscle.

In an advantageous embodiment one or more sensors 20 may comprise sensors 25 configured to measure electrical potential due to eye movement. A suitable sensor is an electrooculography (EOG) sensor. In an advantageous embodiment, as shown in FIG. 4a, there are four sensors that may be arranged in operational proximity to the eye of the user. However, it will be appreciated that other numbers of sensors may be used. In an advantageous embodiment the sensors 25 are conveniently connected to a display unit support 36 of the head set, for example they are affixed thereto or embedded therein.

The sensors 20 may alternatively or additionally comprise one or more of the following sensors: electrocorticogram (ECOG); electrocardiogram (ECG); galvanic skin response (GSR) sensor; respiration sensor; pulse-oximetry sensor; temperature sensor; single unit and multi-unit recording chips for measuring neuron response using a microelectrode system. It will be appreciated that sensors 20 may be invasive (for example ECOG, single unit and multi-unit recording chips) or non-invasive (for example EEG). Pulse-oximetry sensor is used for monitoring a user's oxygen saturation, usually placed on finger tip, and may be used to monitor the status of the user. It will be appreciated that for an embodiment with ECG and/or respiration sensors, the information provided by the sensors may be processes to enable tracking of progress of a user. The information may also be processed in combination with EEG information to predict events corresponding to a state of the user, such as the movement of a body part of the user prior to movement occurring. It will be appreciated that for an embodiment with GSR sensors, the information provided by the sensors may be processed to give an indication of an emotional state of a user. For example, the information may be used during the appended example to measure the level of motivation of a user during the task.

In an advantageous embodiment the physiological parameter sensing system 14 comprises a wireless transceiver which is operable to wirelessly transfer data sensory data to a wireless transceiver of the physiological parameter processing module 54. In this way the head set 18 is convenient to use since there are no obstructions caused by a wired connection.

Referring to FIGS. 4a and 4b, the position/motion detection system 16 comprises one or more sensors 26 suitable for tracking motion of the skeletal structure or a user, or part of the skeletal structure such as an arm. In an advantageous embodiment the sensors comprise one or more cameras which may be arranged separate from the user or attached to the head set 18. Each camera is arranged to capture the movement of a user and pass the image stream to a skeletal tracking module which will be described in more detail in the following.

In an advantageous embodiment the sensors 26 comprise three cameras: two color cameras 28a, 28b and a depth sensor camera 30. However, in an alternative embodiment there is one color camera 28 and a depth sensor 30. A suitable color camera may have a resolution of VGA 640×480 pixels and a frame rate of at least 60 frames per second. The field of view of the camera may also be matched to that of the head mounted display, as will be discussed in more detail in the following. A suitable depth camera may have a resolution of QQ VGA 160×120 pixels. For example, a suitable device which comprises a color camera and a depth sensor is the Microsoft Kinect Suitable color cameras also include models from Aptina Imaging Corporation such as the AR or MT series.

In an advantageous embodiment two color cameras 28a and 28b and the depth sensor 30 are arranged on a display unit support 36 of the head set 18 (which is discussed in more detail below) as shown in FIG. 4. The color cameras 28a, 28b may be arranged over the eyes of the user such that they are spaced apart, for example, by the distance between the pupil axes of a user which is about 65 mm. Such an arrangement enables a stereoscopic display to be captured and thus recreated in VR as will be discussed in more detail in the following. The depth sensor 30 may be arranged between the two cameras 28a, 28b.

In an advantageous embodiment the position/motion detection system 14, sensing unit 14 comprises a wireless transceiver which is operable to wirelessly transfer data sensory data to a wireless transceiver of the skeletal tracking module 52. In this way the head set 18 is convenient to use since there are no obstructions caused by a wired connection.

Referring to FIG. 4a, the head set 18 comprises a display unit 32 having a display means 34a, 34b for conveying visual information to the user. In an advantageous embodiment the display means 34 comprises a head-up display, which is mounted on an inner side of the display unit in front of the eyes of the user so that the user does not need to adjust their gaze to see the information displayed thereon. The head-up display may comprise a non-transparent screen, such an LCD or LED screen for providing a full VR environment. Alternatively, it may comprise a transparent screen, such that the user can see through the display while data is displayed on it. Such a display is advantageous in providing an augmented reality AR. There may be two displays 34a, 34b one for each eye as shown in the figure, or there may be a single display which is visible by both eyes. The display unit may comprise a 2D or 3D display which may be a stereoscopic display. Although the system is described herein as providing a VR image to a user, it will be appreciated that in other embodiments the image be an augmented reality image, mixed reality image, or video image.

In the example of FIG. 4a, the display unit 32 is attached to a display unit support 36. The display unit support 36 supports the display unit 32 on the user and provides a removable support for the headset 18 on the user. In the example, the display unit support 36 extends from proximate the eyes and around the head of the user and is in the form of a pair of goggles as best seen in FIGS. 4a and 4b.

In an alternative embodiment, the display unit 32 is separate from the head set. For example, the display means 34 comprises a monitor or TV display screen or a projector and projector screen.

In an advantageous embodiment part or all of the physiological parameter sensing system 14 and display unit 32 are formed as an integrated part of the head set 18. The cranial sensor support 27 may be connected to the display unit support 36 by a removable attachment (such as a stud and hole attachment, or spring clip attachment) or permanent attachment (such an integrally molded connection or a welded connection or a sewn connection). Advantageously, the head mounted components of the system 10 are convenient to wear and can be easily attached and removed from a user. In the example of FIG. 4a, the strap 27a is connected to the support 36 proximate the ears of the user by a stud and hole attachment. In the example of FIG. 4b, the cap 27c is connected to the support 36 around the periphery of the cap by a sewn connection.

In an advantageous embodiment the system 10 comprises a head movement sensing unit 40. The head movement sensing unit comprises a movement sensing unit 42 for tracking head movement of a user as they move their head during operation of the system 10. The head movement sensing unit 42 is configured to provide data in relation to the X, Y, Z coordinate location and the roll, pitch, and yaw of a head of a user. This data is provided to a head tracking module, which is discussed in more detail in the following, and processes the data such that the display unit 32 can update the displayed VR images in accordance with head movement. For example, as the user moves their head to look to the left the displayed VR images move to the left. While such an operation is not essential it is advantageous in providing a more immersive VR environment. In order to maintain realism, it has been found that the maximum latency of the loop defined by movement sensed by the head movement sensing unit 42 and the updated VR image is 20 ms.

In an advantageous embodiment, the head movement sensing unit 42 comprises an acceleration sensing means 44, such as an accelerometer configured to measure acceleration of the head. In an advantageous embodiment, the sensor 44 comprises three in-plane accelerometers, wherein each in-plane accelerometer is arranged to be sensitive to acceleration along a separate perpendicular plate. In this way, the sensor is operable to measure acceleration in three-dimensions. However, it will be appreciated that other accelerometer arrangements are possible. For example, there may only be two in-plane accelerometers arranged to be sensitive to acceleration along separate perpendicular plates such that two-dimensional acceleration is measured. Suitable accelerometers include piezoelectric, piezoresistive, and capacitive variants. An example of a suitable accelerometer is the Xsens Technologies BV MTi 10-series sensor.

In an advantageous embodiment, the head movement sensing unit 42 further comprises a head orientation sensing means 47 which is operable to provide data in relation to the orientation of the head. Examples of suitable head orientation sensing means include a gyroscope and a magnetometer 48 which are configured to measure the orientation of a head of a user.

In an advantageous embodiment, the head movement sensing unit 42 may be arranged on the headset 18. For example, the movement sensing unit 42 may be housed in a movement sensing unit support 50 that is formed integrally with or is attached to the cranial sensor support 27 and/or the display unit support 36 as shown in FIGS. 4a and 4b.

In an advantageous embodiment, the system 10 comprises an eye gaze sensing unit 100. The eye gaze sensing unit 100 comprises one or more eye gaze sensors 102 or sensing the direction of gaze of the user. In an advantageous embodiment, the eye gaze sensor 102 comprises one or more cameras arranged in operation proximity to one or both eyes of the user. Each camera 102 may be configured to track eye gaze by using the center of the pupil and infrared/near-infrared non-collimated light to create corneal reflections (CR). However, it will be appreciated that other sensing means may be used such as electrooculogram (EOG) or eye-attached tracking. The data from the movement sensing unit 42 is provided to an eye tracking module, which is discussed in more detail in the following, and processes the data such that the display unit 32 can update the displayed VR images in accordance with eye movement. For example, as the user moves their eyes to look to the left, the displayed VR images pan to the left. While such an operation is not essential, it is advantageous in providing a more immersive VR environment. In order to maintain realism, it has been found that the maximum latency of the loop defined by movement sensed by the eye gaze sensing unit 100 and the updated VR image is about 50 ms, however in an advantageous embodiment it is 20 ms or lower.

In an advantageous embodiment, the eye gaze sensing unit 100 may be arranged on the headset 18. For example, the eye gaze sensing unit 42 may be attached to the display unit support 36 as shown in FIG. 4a.

The control system 12 processes data from the physiological parameter sensing system 14 and the position/motion detection system 16, and optionally one or both of the head movement sensing unit 40 and the eye gaze sensing module 100, together with operator input data supplied to an input unit, to generate VR (or AR) data which is displayed by the display unit 32. To perform such a function, in the advantageous embodiment shown in FIGS. 1 and 2, the control system 12 may be organized into a number of modules, such as: a skeletal tracking module 52; a physiological parameter processing module 54; a VR generation module 58; a head tracking module 58; and an eye gaze tracking module 100 which are discussed in the following.

The skeletal tracking module 52 processes the sensory data from the position/motion detection system 16 to obtain joint position/movement data for the VR generation module 58. In an advantageous embodiment, the skeletal tracking module 52, as shown in FIG. 3b, comprises a calibration unit 60, a data fusion unit 62, and a skeletal tracking unit 64, the operations of which will now be discussed.

The sensors 26 of the position/motion detection system 16 provide data in relation to the position/movement of a whole or part of a skeletal structure of a user to the data fusion unit 62. The data may also comprise information in relation to the environment, for example the size and arrangement of the room the user is in. In the exemplary embodiment, wherein the sensors 26 comprise a depth sensor 30 and a color cameras 28a, 28b the data comprises color and depth pixel information.

The data fusion unit 62 uses this data, and the calibration unit 62, to generate a 3D point cloud comprising a 3D point model of an external surface of the user and environment. The calibration unit 62 comprises data in relation to the calibration parameters of the sensors 26 and a data matching algorithm. For example, the calibration parameters may comprise data in relation to the deformation of the optical elements in the cameras, color calibration and hot and dark pixel discarding and interpolation. The data matching algorithm may be operable to match the color image from cameras 28a and 28b to estimate a depth map which is referenced with respect to a depth map generated from the depth sensor 30. The generated 3D point cloud comprises an array of pixels with an estimated depth such that they can be represented in a three-dimensional coordinate system. The color of the pixels is also estimated and retained.

The data fusion unit 62 supplies data comprising 3D point cloud information, with pixel color information, together with color images to the skeletal tracking unit 64. The skeletal tracking unit 64 processes this data to calculate the position of the skeleton of the user and therefrom estimate the 3D joint positions. In an advantageous embodiment, to achieve this operation, the skeletal tracking unit can be organized into several operational blocks, for example: 1) segment the user from the environment using the 3D point cloud data and color images; 2) detect the head and body parts of the user from the color images; 3) retrieve a skeleton model of the user from 3D point cloud data; and 4) use inverse kinematic algorithms together with the skeleton model to improve joint position estimation. The skeletal tracking unit 64 outputs the joint position data to the VR generation module 58 which is discussed in more detail in the following. The joint position data is time stamped by a clock module such that the motion of a body part can be calculated by processing the joint position data over a given time period.

Referring to FIGS. 2 and 3, the physiological parameter processing module 54 processes the sensory data from the physiological parameter sensing system 14 to provide data which is used by the VR generation module 58. The processed data may, for example, comprise information in relation to the intent of a user to move a particular body part or a cognitive state of a user (for example, the cognitive state in response to moving a particular body part or the perceived motion of a body part). The processed data can be used to track the cognitive state of the user, for example, as part of a study to determine user reaction to certain audio or visual stimulation and the like as discussed further below.

The cortical activity is measured and recorded as the user performs specific body part movements/intended movements, which are instructed in the VR environment. Examples of such instructed movements are provided in the appended examples. To measure the cortical activity, the EEG sensors 22 are used to extract event related electrical potentials and event related spectral perturbations, in response to the execution and/or observation of the movements/intended movements which can be viewed in VR as an avatar of the user.

For example the following bands provide data in relation to various operations: slow cortical potentials (SCPs), which are in the range of 0.1-1.5 Hz and occur in motor areas of the brain provide data in relation to preparation for movement; mu-rhythm (8-12 Hz) in the sensory motor areas of the brain provide data in relation to the execution, observation and imagination of movement of a body part; beta oscillations (13-30 Hz) provide data in relation to sensory motor integration and movement preparation. It will be appreciated that one or more of the above potentials or other suitable potentials may be monitored. Monitoring such potentials over a period of time can be used to provide information in relation to the recovery or a user.

Referring to FIG. 5, an advantageous exemplary arrangement of sensors 20 is provided which is suitable for measuring neural events as a user performs various sensorimotor and/or cognitive tasks or senses various stimuli (e.g., visual stimuli, audio stimuli, and the like). EOG sensors 25 are advantageously arranged to measure eye movement signals. In this way the eye movement signals can be isolated and accounted for when processing the signals of other groups to avoid contamination. EEG sensors 22 may advantageously be arranged into groups to measure motor areas in one or more areas of the brain, for example: central (C1-C6, Cz); fronto-central (FC1-FC4, FCZ); centro-pariental (CP3, CP4, CPZ). In an advantageous embodiment contralateral EEG sensors C1, C2, C3 and C4 are arranged to measure arm/hand movements. The central, fronto-central, and centro-pariental sensors may be used for measuring SCPs.

In an advantageous embodiment, the physiological parameter processing module 54 comprises a re-referencing unit 66 which is arranged to receive data from the physiological parameter sensing system 14 and configured to process the data to reduce the effect of external noise on the data. For example, it may process data from one or more of the EEG, EOG, or EMG sensors. The re-referencing unit 66 may comprise one or more re-referencing blocks: examples of suitable re-referencing blocks include mastoid electrode average reference, and common average reference. In the example embodiment a mastoid electrode average reference is applied to some of the sensors and common average reference is applied to all of the sensors. However, it will be appreciated that other suitable noise filtering techniques may be applied to various sensors and sensor groups.

In an advantageous embodiment, the processed data of the re-referencing unit 66 may be output to a filtering unit 68. In an embodiment wherein there is no re-referencing unit, the data from the physiological parameter sensing system 14 is fed directly to the filtering unit 68, however. The filtering unit 68 may comprise a spectral filtering module 70 which is configured to band pass filter the data for one or more of the EEG, EOG, and EMG sensors. With respect to the EEG sensors, in an advantageous embodiment, the data is band-pass filtered for one or more of the sensors to obtain the activity on one or more of the bands: SCPs, theta, alpha, beta, gamma, mu, gamma, delta. In an advantageous embodiment, the bands SCPs (0.1-1.5 Hz), alpha and mu (8-12 Hz), beta (18-30 Hz) delta (1.5-3.5 Hz), theta (3-8 Hz) and gamma (30-100 Hz) are filtered for all of the EEG sensors. With respect to EMG and EOG sensors, similar spectral filtering may be applied but with different spectral filtering parameters. For example, for EMG sensors spectral filtering of a 30 Hz high pass cut off may be applied.

The filtering unit 68 may alternatively or additionally comprise a spatial filtering module 72. In an advantageous embodiment, a spatial filtering module 72 is applied to the SCPs band data from the EEG sensors (which is extracted by the spectral filtering module 70), however it may also be applied to other extracted bands. A suitable form of spatial filtering is spatial smoothing which comprises weighted averaging of neighboring electrodes to reduce spatial variability of the data. Spatial filtering may also be applied to data from the EOG and EMG sensors.

The filtering unit 68 may alternatively or additionally comprise a Laplacian filtering module 74, which is generally for data from the EEG sensors but may also be applied to data from the EOG and EMG sensors. In an advantageous embodiment, a Laplacian filtering module 72 is applied to each of the Alpha, Mu, and Beta band data of the EEG sensors which is extracted by the spectral filtering module 70. However, it may be applied to other bands. The Laplacian filtering module 72 is configured to further reduce noise and increase spatial resolution of the data.

The physiological parameter sensing system 14 may further comprise an event marking unit 76. In an advantageous embodiment, when the physiological parameter sensing system 14 comprises a re-referencing unit and/or a filtering unit 68, the event marking unit 76 is arranged to receive processed data from either or both of these units when arranged in series (as shown in the embodiment of FIG. 3c). The event marking unit 76 is operable to use event-based markers determined by an exercise logic unit (which will be discussed in more detail in the following) to extract segments of sensory data. For example, when a specific instruction to move a body part is sent to the user from the exercise logic unit, a segment of data is extracted within a suitable time frame following the instruction. The data may, in the example of an EEG sensor, comprise data from a particular cortical area to thereby measure the response of the user to the instruction. For example, an instruction may be sent to the user to move their arm and the extracted data segment may comprise the cortical activity for a period of 2 seconds following instruction. Other example events may comprise the following: potentials in response to infrequent stimuli in the central and centro-parietal electrodes; movement related potentials that are central SCPs (slow cortical potentials) which appear slightly prior to movement; and error related potentials.

In an advantageous embodiment, the event marking unit 76 is configured to perform one or more of following operations: extract event-related potential data segments from the SCP band data; extract event related spectral perturbation marker data segments from alpha and beta or mu or gamma band data; extract spontaneous data segments from beta band data. In the aforementioned, spontaneous data segments correspond to EEG segments without an event marker, and are different to event related potentials, the extraction of which depends on the temporal location of the event marker.

The physiological parameter sensing system 14 may further comprise an artefact detection unit 78 which is arranged to receive the extracted data segments from the event marking unit 76 and is operable to further process the data segments to identify specific artefacts in the segments. For example, the identified artefacts may comprise 1) movement artefacts: the effect of a user movement on a sensor/sensor group; 2) electrical interference artefacts: interference, typically 50 Hz, from the mains electrical supply; 3) eye movement artefacts: such artefacts can be identified by the EOG sensors 25 of the physiological parameter sensing system 14; and the like. In an advantageous embodiment, the artefact detection unit 78 comprises an artefact detector module 80 which is configured to detect specific artefacts in the data segments. Such data segments can include, for example, an erroneous segment which requires deleting or a portion of the segment which is erroneous and requires removing from the segment. The advantageous embodiment further comprises an artefact removal module 82, which is arranged to receive the data segments from the event marking unit 76 and artefact detected from the artefact detector module 80 to perform an operation of removing the detected artefact from the data segment. Such an operation may comprise a statistical method such as a regression model which is operable to remove the artefact from the data segment without loss of the segment. The resulting data segment is thereafter output to the VR generation module 58, wherein it may be processed to provide real-time VR feedback which may be based on movement intention as will be discussed in the following. The data may also be stored to enable the progress of a user to be tracked.

In embodiments comprising other sensors, such as ECG, respiration sensors and GSR sensors, it will be appreciated that the data from such sensors can be processed using one of more of the above-mentioned techniques where applicable, for example: noise reduction; filtering; event marking to extract event relate data segments; artefact removal from extracted data segments; and the like.

The head tracking module 56 is configured to process the data from the head movement sensing unit 40 to determine the degree of head movement. The processed data is sent to the VR generation module 58, wherein it is processed to provide real-time VR feedback to recreate the associated head movement in the VR environment. For example, as the user moves their head to look to the left the displayed VR images move to the left.

The eye gaze tracking module 104 is configured to process the data from the eye gaze sensing unit 100 to determine a change in gaze of the user. The processed data is sent to the VR generation module 58, wherein it is processed to provide real-time VR feedback to recreate the change in gaze in the VR environment.

Referring now to FIG. 3b, the VR generation module 58 is arranged to receive data from the skeletal tracking module 52, physiological parameter processing module 54, and optionally one or both of the head tracking module 56 and the eye gaze tracking module 104; and is configured to process this data such that it is contextualized with respect to a status of an exercise logic unit (which is discussed in more detail in the following), and to generate a VR environment based on the processed data.

In an advantageous embodiment the VR generation module 58 may be organized into several units: an exercise logic unit 84; a VR environment unit 86; a body model unit 88; an avatar posture generation unit 90; a VR content integration unit 92; an audio generation unit 94; and a feedback generation unit 96. The operation of these units will now be discussed.

In an advantageous embodiment, the exercise logic unit 84 is operable to interface with a user input, such as a keyboard or other suitable input device. The user input may be used to select a particular task from a library of tasks and/or set particular parameters for a task. The appended example provides details of such a task.

In an advantageous embodiment, a body model unit 88 is arranged to receive data from the exercise logic unit 84 in relation to the particular part of the body required for the selected task. For example, this may comprise the entire skeletal structure of the body or a particular part of the body such as an arm. The body model unit 88 thereafter retrieves a model of the required body part, for example from a library of body parts. The model may comprise a 3D point cloud model, or other suitable model.

The avatar posture generation unit 90 is configured to generate an avatar based on the model of the body part from the body part model 88.

In an advantageous embodiment, the VR environment unit 86 is arranged to receive data from the exercise logic unit 84 in relation to the particular objects which are required for the selected task. For example, the objects may comprise a disk or ball to be displayed to the user.

The VR content integration unit may be arranged to receive the avatar data from the avatar posture generation unit 90 and the environment data from the VR environment unit 86 and to integrate the data in a VR environment. The integrated data is thereafter transferred to the exercise logic unit 58 and also output to the feedback generation unit 86. The feedback generation unit 86 is arranged to output the VR environment data to the display means 34 of the headset 18.

During operation of the task the exercise logic unit 84 receives data comprising joint position information from the skeletal tracking module 64, data comprising physiological data segments from the physiological parameter processing module 54 data from the body model unit 88 and data from the VR environment unit 86. The exercise logic unit 84 is operable to processes the joint position information data which is in turn sent to the avatar posture generation unit 90 for further processing and subsequent display. The exercise logic unit 84 may optionally manipulated the data so that it may be used to provide VR feedback to the user. Examples of such processing and manipulation include amplification of erroneous movement; auto correction of movement to induce positive reinforcement; mapping of movements of one limb to another; and the like.

As the user moves, interactions and/or collisions with the objects, as defined by the VR environment unit 86, in the VR environment, are detected by the exercise logic unit 84 to further update the feedback provided to the user.

The exercise logic unit 84 may also provide audio feedback. For example, an audio generation unit (not shown) may receive audio data from the exercise logic unit, which is subsequently processed by the feedback unit 94 and output to the user, for example, by headphones (not shown) mounted to the headset 18. The audio data may be synchronized with the visual feedback, for example, to better indicate collisions with objects in the VR environment and to provide a more immersive VR environment.

In an advantageous embodiment, the exercise logic unit 84 may send instructions to the physiological parameter sensing system 14 to provide feedback to the user via one or more of the sensors 20 of the physiological parameter sensing system 14. For example, the EEG 22 and/or EMG 24 sensors may be supplied with an electrical potential that is transferred to the user. With reference to the appended example, such feedback may be provided during the task. For example, at stage 5, wherein there is no arm movement, an electrical potential may be sent to EMG 24 sensors arranged on the arm and/or EEG sensors to attempt to stimulate the user into moving their arm. In another example, such feedback may be provided before initiation of the task, for instance, a set period of time before the task, to attempt to enhance a state of memory and learning.

In an advantageous embodiment, the control system comprises a clock module 106. The clock module may be used to assign time information to the data and various stages of input and output and processing. The time information can be used to ensure the data is processed correctly, for example, data from various sensors is combined at the correct time intervals. This is particularly advantageous to ensure accurate real-time processing of multimodal inputs from the various sensors and to generate real-time feedback to the user. The clock module 106 may be configured to interface with one or more modules of the control system to time stamp data. For example: the clock module 106 interfaces with the skeletal tracking module 52 to time stamp data received from the position/motion detection system 16; the clock module 106 interfaces with the physiological parameter processing module 54 to time stamp data received from the physiological parameter sensing system 14; the clock module 106 interfaces with the head tracking module 58 to time stamp data received from the head movement sensing unit 40; the clock module 106 interfaces with the eye gaze tracking module 104 to time stamp data received from the eye gaze sensing unit 100. Various operations on the VR generation module 58 may also interface with the clock module 106 to time stamp data, for example data output to the display means 34.

Unlike complex conventional systems that connect several independent devices together, in the present invention, synchronization occurs at the source of the data generation (for both sensing and stimulation), thereby ensuring accurate synchronization with minimal latency and, importantly, low jitter. For example, for a stereo head-mounted display with refresh rate of 60 Hz, the delay would be as small as 16.7 ms. This is not presently possible with a combination of conventional stand-alone or independent systems. An important feature of the present invention is that it is able to combine a heterogeneous ensemble of data, synchronizing them into a dedicated system architecture at source for ensuring multimodal feedback with minimal latencies. The wearable compact head mounted device allows easy recording of physiological data from brain and other body parts.

Synchronization Concept:

Latency or Delay (T): It is the time difference between the moment of user's actual action or brain state to the moment of its corresponding feedback/stimulation. It is a positive constant in a typical application. Jitter (AT) is the trial to trial deviation in Latency or Delay. For applications that require for instance immersive VR or AR, both latency T and jitter AT should be minimized to the least possible. Whereas in brain computer interface and offline applications, latency T can be compromised but jitter AT should be as small as possible.

Referring to FIGS. 1a and 1b, two conventional prior-art system architectures are schematically illustrated. In these, the synchronization may be ensured to some degree but jitter (AT) is not fully minimized.

Design-I (FIG. 1a):

In this design, the moment at which a visual cue is supplied to user is registered directly in the computer while acquiring the EEG signal that is acquired via a USB connection or serial connection. Meaning, the computer assumes, the moment at which it is registered with acquired from user's brain is the moment a cue is displayed to the user. Note that there are inherent delays and jitters in this design. First due to the USB/serial port connectivity to computer, the registration of the sample into computer is has nonzero variable latency. Second, the moment the display command is released from the computer, it undergoes various delay due to underlying display driver, graphical processing unit, and signal propagation, which is also not a constant. Hence, these two kinds of delays add up and compromise alignment of visually evoked potentials.

Design-II (FIG. 1b):

To avoid the above problem, it is known to use a photo diode to measure the cue and synchronize its signal directly with an EEG amplifier. In this design, usually a photo-diode is placed on the display to sense a light. Usually, a cue is presented to user at the same time a portion of screen where the photo-diode is attached is lighted up. This way the moment at which the cue is presented is registered with photo-diode and supplied to EEG amplifier. This way EEG and visual cue information are directly synchronized at source. This procedure is accurate for alighting visually evoked trials, however, has a number of drawbacks:
The number of visual cues it can code are limited to number of photodiodes. A typical virtual reality based visual stimulation would have large number of events to be registered together with physiological signals accurately.
The use of photo-diode in a typical micro-display (e.g., 1 square inch size, with pixel density of 800×600) of a head-mounted display would be difficult and even worse reduces usability. Note also that for the photo-diode to function, ample light should be supplied to the diode resulting in a limitation.
The above drawbacks are further complicated when a plurality of stimuli (such as audio, magnetic, electrical, and mechanical) must be synchronized with plurality of sensors data (such as EEG, EMG, ECG, video camera, inertial sensors, respiration sensor, pulse oximetry, galvanic skin potentials, etc.).

In embodiments of the present invention, the above drawbacks are addressed to provide a system that is accurate and scalable to many different sensors and many different stimuli. This is achieved by employing a centralized clock system that supplies a time-stamp information and each sensor's samples are registered in relation to this to the time-stamp.

In an embodiment, each stimulation device may advantageously be equipped with an embedded sensor whose signal is registered by a synchronization device. This way, a controller can interpret plurality of sensor data and stimulation data can be interpreted accurately for further operation of the system.

In an embodiment, in order to reduce the amount of data to synchronize from each sensor, instead of using a real sensor, video content code from a display register may be read.

### Example 1: Operation of System (10) in Exemplary “Reach an Object” Task

In this particular example an object 110, such as a 3D disk, is displayed in a VR environment 112 to a user. The user is instructed to reach to the object using a virtual arm 114 of the user. In the first instance the arm 114 is animated based on data from the skeletal tracking module 16 derived from the sensors of the position/motion detection system 16. In the second instance, wherein there is negligible or no movement detected by the skeletal tracking module 16, then the movement is based data relating to intended movement from the physiological parameter processing module 52 detected by the physiological parameter sensing system 14, and in particular the data may be from the EEG sensors 22 and/or EMG sensors 24.

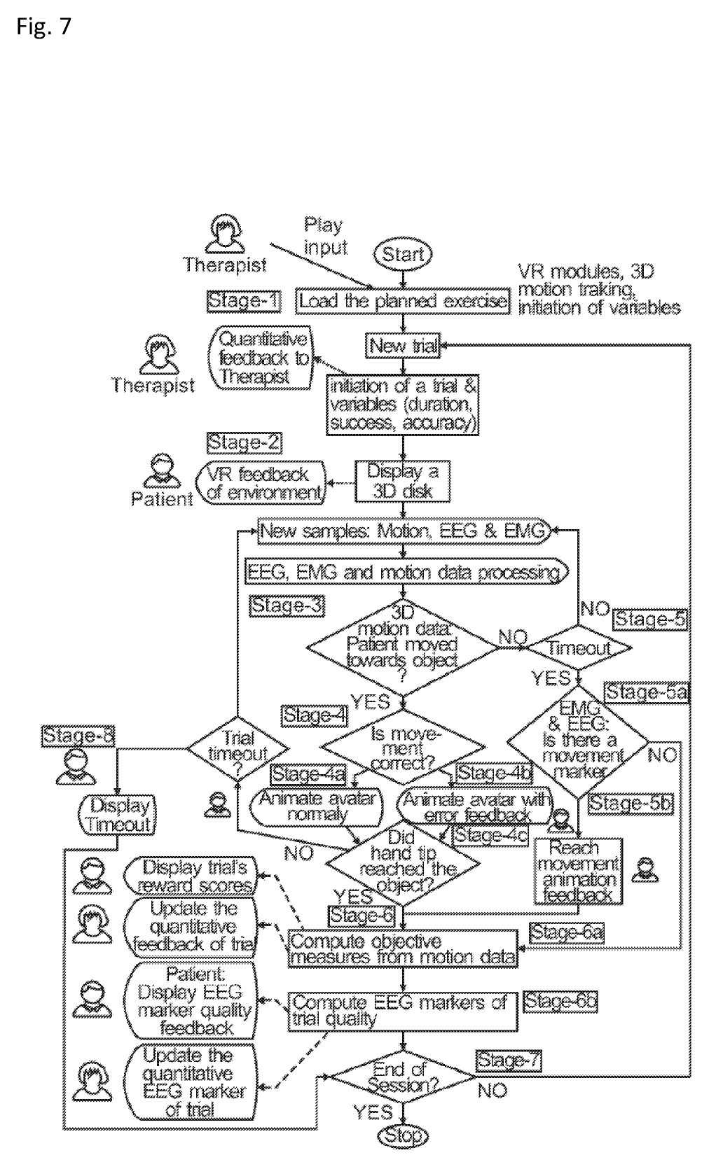

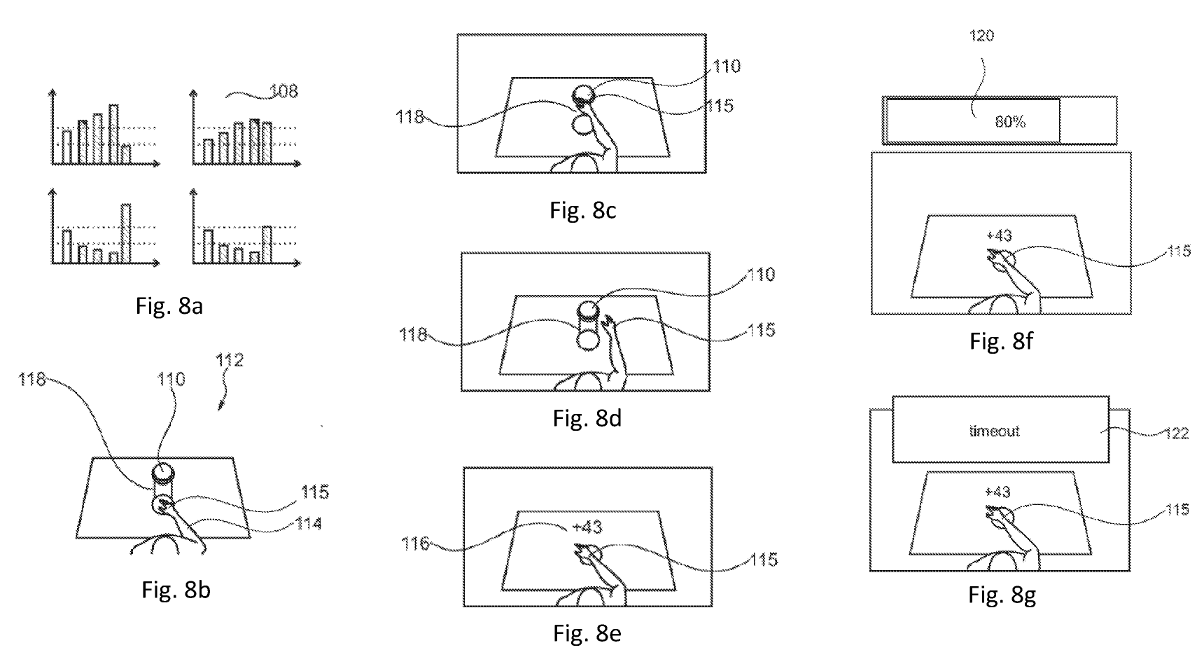

FIGS. 7 and 8a-8g describe the process in more detail. At stage 1 in FIG. 7, a user, such as an end user or operator, interfaces with a user input of the exercise logic unit 84 of the VR generation module 58 to select a task from a library of tasks which may be stored. In this example, a ‘reach an object task’ is selected. At this stage, the user may be provided with the results 108 of previous like tasks, as shown in FIG. 8a. These results may be provided to aid in the selection of the particular task or task difficulty. The user may also input parameters to adjust the difficulty of the task, for example based on a level of success from the previous task.

At stage 2, the exercise logic unit 84 initializes the task. This comprises steps of the exercise logic unit 84 interfacing with the VR environment unit 86 to retrieve the parts (such as the disk 110) associated with the selected task from a library of parts. The exercise logic unit 84 also interfaces with the body model unit 88 to retrieve, from a library of body parts, a 3D point cloud model of the body part (in this example a single arm 114) associated with the exercise. The body part data is then supplied to the avatar posture generation unit 90 so that an avatar of the body part 114 can be created. The VR content integration unit 92 receives data in relation to the avatar of the body part and parts in the VR environment and integrates them in a VR environment. This data is thereafter received by the exercise logic unit 84 and is output to the display means 34 of the headset 18 as shown in FIG. 8b. The target path 118 for the user to move a hand 115 of the arm 114 along is indicated, for example, by coloring it blue.

At stage 3, the exercise logic unit 84 interrogates the skeletal tracking module 16 to determine whether any arm movement has occurred. The arm movement being derived from the sensors of the position/motion detection system 16 which are worn by the user. If a negligible amount of movement (for example, an amount less than a predetermined amount, which may be determined by the state of the user and location of movement) or no movement has occurred then stage 5 is executed, else stage 4 is executed.

At stage 4 the exercise logic unit 84 processes the movement data to determine whether the movement is correct. If the user has moved their hand 115 in the correct direction, for example, towards the object 110, along the target path 118, then stage 4a is executed and the color of the target path may change, for example it is colored green, as shown in FIG. 8c. Else, if the user moves their hand 115 in an incorrect direction, for example, away from the object 110, Then stage 4b is executed and the color of the target path may change, for example it is colored red, as shown as FIG. 8d.

Following stage 4a and 4b stage 4c is executed, wherein the exercise logic unit 84 determines whether the hand 115 has reached the object 110. If the hand has reached the object, as shown in FIG. 8e then stage 6 is executed, else stage 3 is re-executed.

At stage 5 the exercise logic unit 84 interrogates the physiological parameter processing module 52 to determine whether any physiological activity has occurred. The physiological activity is derived from the sensors of the physiological parameter sensing system module 14, which are worn by the user, for example the EEG and/or EMG sensors. EEG and EMG sensors may be combined to improve detection rates, and in the absence of a signal from one type of sensor a signal from the other type of sensor maybe used. If there is such activity, then it may be processed by the exercise logic unit 84 and correlated to a movement of the hand 115. For example, a characteristic of the event related data segment from the physiological parameter processing module 52, such as the intensity or duration of part of the signal, may be used to calculate a magnitude of the hand movement 115. Thereafter stage 6 is executed.

At stage 6a, if the user has successfully completed the task, then to provide feedback 116 to the user a reward score may be calculated, which may be based on the accuracy of the calculated trajectory of the hand 115 movement. FIG. 8e shows the feedback 116 displayed to the user. The results from the previous task may also be updated.

Thereafter, stage 6b is executed, wherein a marker strength of the sensors of the physiological parameter sensing system module 14, for example the EEG and EMG, sensors may be used to provide feedback 118. FIG. 8f shows an example of the feedback 120 displayed to the user, wherein the marker strength is displayed as a percentage of a maximum value. The results from the previous task may also be updated. Thereafter, stage 7 is executed, wherein the task is terminated.

As stage 8, if there is no data provided by either of the sensors of the physiological parameter sensing system module 14 or the sensors of the position/motion detection system 16 with in a set period of time then time out 122 occurs, as shown in FIG. 8g and stage 7 is executed.

### Example 2: Hybrid Brain Computer Interface with Virtual Reality Feedback with Head-Mounted Display, Robotic System, and Functional Electrical Stimulation

The physical embodiment illustrated in FIG. 9, comprises a wearable system having a head-mounted display (HMD) 18 to display virtual reality 3D video content on micro-displays (e.g., in first-person perspective), a stereo video camera 30, and a depth camera 28, whose data is used for tracking the wearer's own arm, objects, and any second person under the field of view (motion tracking unit). Additionally, the EEG electrodes 22 placed over the head of the wearer 1, EMG electrodes 24 placed on the arm will measure electrical activity of the brain and of muscles respectively, used for inferring user's intention in making a goal directed movement. Additionally, there exists an Inertial Measurement Unit (IMU) 29 that is used for tracking head movements. The executed or intended movements are rendered in the virtual reality display. In case of evidence of the movements through the biological sensor data (i.e., EEG, EMG, and motion tracing) feedback mechanisms aid the user in making goal directed movement using a robotic system 41. Furthermore, functional electrical stimulation (FES) system 31 activates muscles of the arm in completing the planned movement. Additionally, the feedback mechanisms shall provide appropriate stimulation tightly coupling to the intention to move to ensure implementation of a Hebbian learning mechanism. In the following text we describe an architecture that implements high quality synchronization of sensor data with stimulation data.

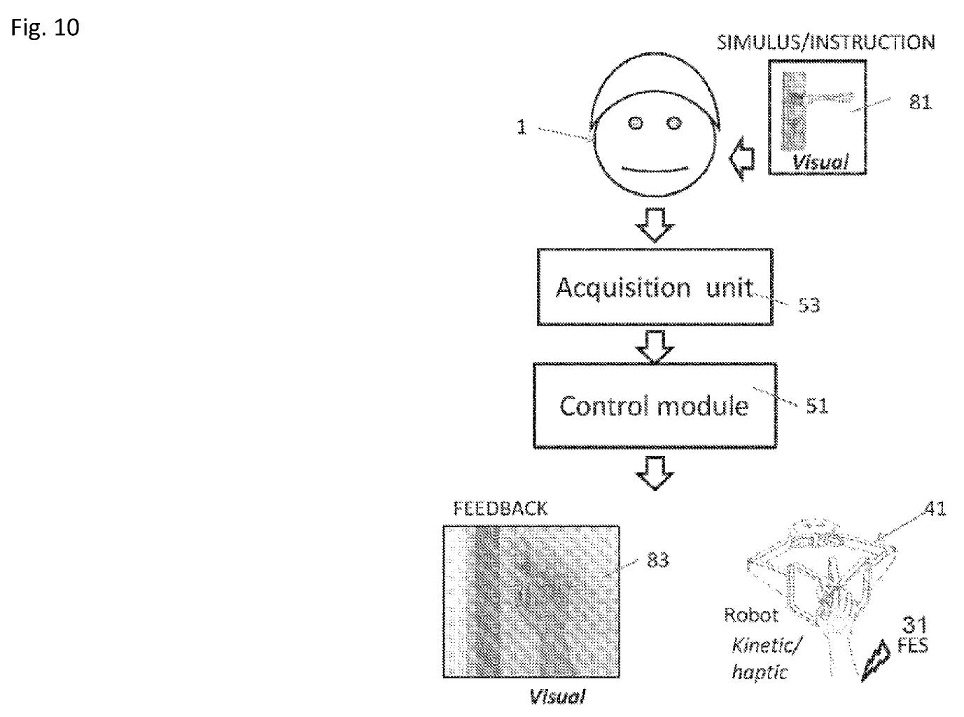

The following paragraph describes a typical trial in performing a typical goal directed task, which could be repeated by the user several times to complete a typical training session. As shown in FIG. 10, a 3D visual cue 81, in this case a door knob, when displayed in the HMD could instruct the user to make a movement corresponding to opening the door. Followed by the visual cue, the user may attempt to make the suggested movement. Sensor data (EEG, EMG, IMU, motion data) is acquired in synchronization with the moment of presentation of the visual cue. The control system 51 then extracts the sensor data and infers user intention and a consensus is made in providing feedback to the user through a robot 41 that moves the arm, and HMD displays movement of an avatar 83, which is animated based on the inferred data. A Functional Electrical Stimulation (FES) 31 is also synchronized together with other feedbacks ensuring a congruence among them.

An exemplary architecture of this system is illustrated in FIG. 2d. The acquisition unit 53 acquires physiological data (i.e., EEG 22, EMG 24, IMU 29, and camera system 30). The camera system data include stereo video frames and depth sensor data. Additionally, the stimulation related data such as the moment at which a particular image frame of the video is displayed on the HMD, robot's motor data and sensors 23 and that of FES 31 stimulation data are also sampled by the acquisition unit 53. This unit associates each sensor and stimulation sample with a time stamp (TS) obtained from the clock input. The synchronized data is then processed by control system and is used in generating appropriate feedback content to the user through VR HMD display, robotic movement as well as FES stimulation.

Inputs of the System

Inertial measurement unit (IMU) sensors 29, for instance including an accelerometer, a gyroscope, a magneto-meter: Purpose, to track head movements. This data is used for rendering VR content as well as to segment EEG data where the data quality might be degraded due to movement. Camera system 30, 28: The camera system comprises a stereo camera 30, and a depth sensor 28. The data of these two sensors are combined to compute tracking data of a wearer's own movements of upper limbs, and for tracking wearer's own arm movements. These movements are then used in animating the avatar in the virtual reality on micro displays 32 and in detecting if there was a goal directed movements, which is then used for triggering feedback through display 32, robot 41, and stimulation device FES 31. Sensors EEG 22 and EMG 24 are used for inferring if there was an intention to make a goal directed movement.

Outputs of the System/Feedback Systems
Micro-displays 34 of headset 18: Renders 2D/3D virtual reality content, where a wearer experiences the first-person perspective of the virtual world as well as of his own avatar with its arms moving in relation to his own movements.
Robotic system 41: Robotic system described in this invention is used for driving movements of the arm, where the user holds a haptic knob. The system provides a range of movements as well as haptic feedback of natural movements of activities of daily living.
Functional Electrical Stimulation (FES) device 31: Adhesive electrodes of FES system are placed on user's arms to stimulate nerves, which up on activated can restore the lost voluntary movements of the arm. Additionally, the resulting movements of the hand results in kinesthetic feedback to the brain.

Data Processing

The following paragraphs describe the data manipulations from inputs till outputs.

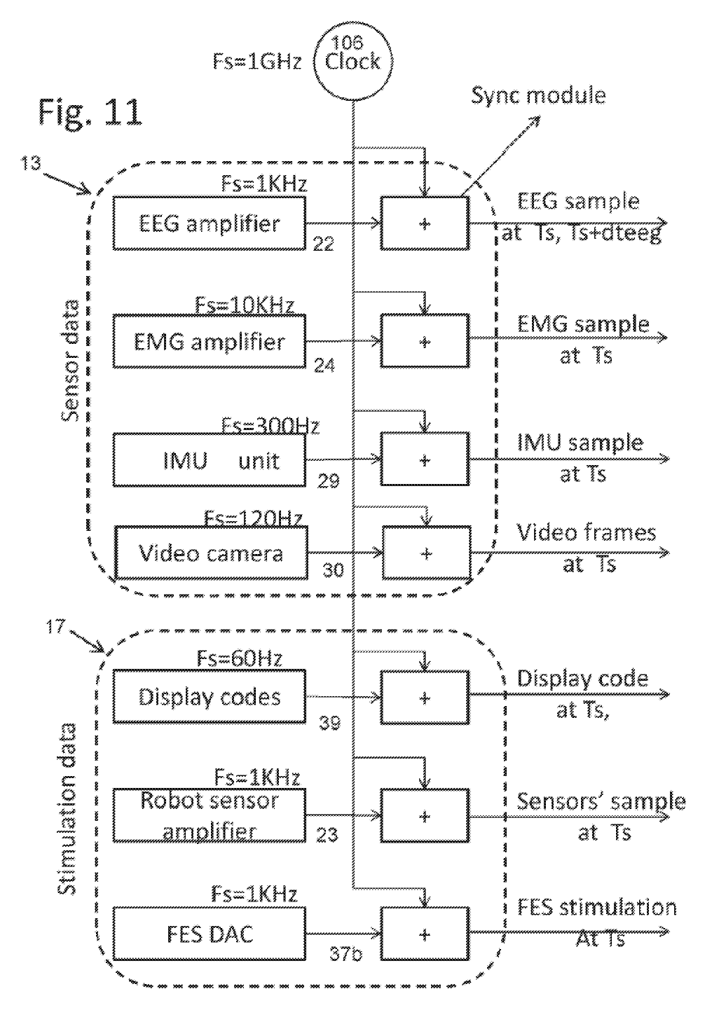

Acquisition Unit 53: The description of acquisition unit 53 ensures near perfect synchronization of inputs/sensor data and outputs/stimulation/feedback of the system as illustrated in the FIG. 11. Each sensor data may have different sampling frequency and whose sampling may have not initiated at exact same moment due to non-shared internal clock. In this example, the sampling frequency of EEG data is 1 kHz, EMG data is 10 KHz, IMU data is 300 Hz, and video camera data is 120 frames per second (fps). Similarly, the stimulation signals have different frequencies, where the display refresh rate is at 60 Hz, robot sensors of 1 KHz, and FES data at 1 KHz.

The acquisition unit 53 aims at solving the issue of synchronization of inputs and outputs accurately. In achieving so, the outputs of the system are sensed either with dedicated sensors or indirectly recorded from a stage before stimulation, for instance as follows:
Sensing the micro-display: Generally, the video content that is generated in the control system is first pushed to a display register 35 (a final stage before the video content is activated on the display). Together with video content, the controller sends a code to a part of the register (say N bits) corresponding to one or more pixels (not too many pixels, so that the user is not disturbed). The corner pixels in the micro display are preferred as they may not be visible to user. The codes (a total of 2 N) may be defined by the controller or the exercise logic unit describing the display content.
Sensing FES: The FES data can be red from its last stage of generation, i.e., from the DAC.
Sensing Robot's movements: The robots motors are embedded with sensors providing information on angular displacement, torque, and other control parameters of the motors.

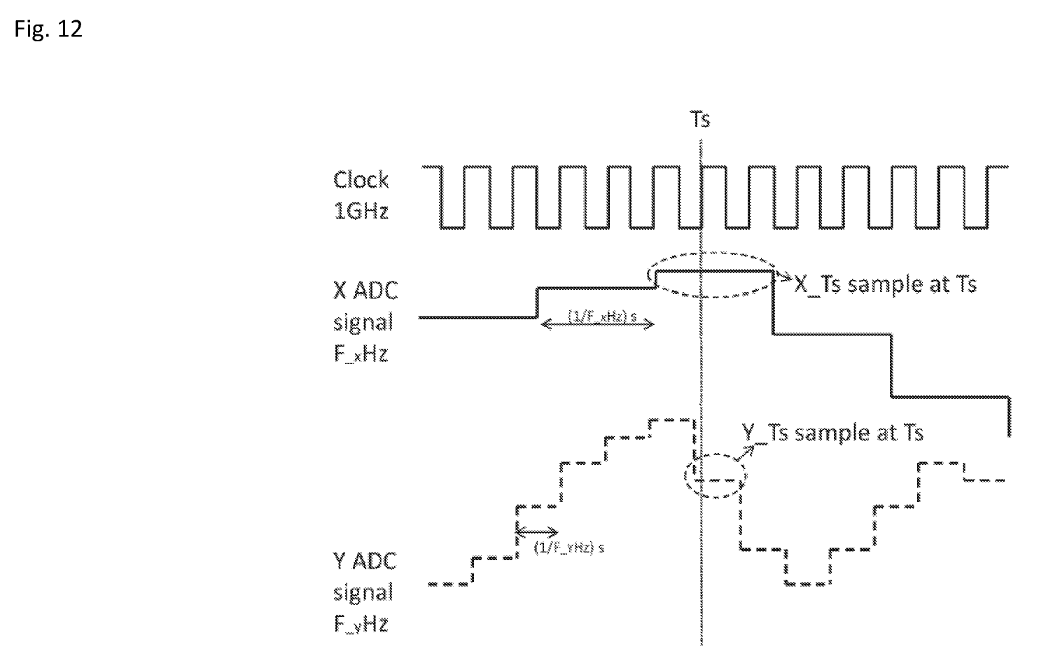

Now using a clock signal with preferably a much higher frequency than that of the inputs and outputs (e.g., 1 GHz), but at least double the highest sampling frequency among sensors and stimulation units, the acquisition module 53 reads the sensor samples and attaches a time stamp as illustrated in the FIG. 12. When a sample of a sensor arrives from its ADC 37a, its time of arrival is annotated with next immediate rising edge of the clock signal. Similarly, for every sensor and stimulation data a time-stamp is associated. When these samples arrive at the controller, it interprets the samples according to the time stamp of arrival leading to minimized jitters across sensors and stimulations.

Physiological Data Analysis

The physiological data signals EEG and EMG are noisy electrical signals and preferably are pre-processed using appropriate statistical methods. Additionally, the noise can also be reduced by better synchronizing the events of stimulation and behavior with the physiological data measurements with negligible jitter.

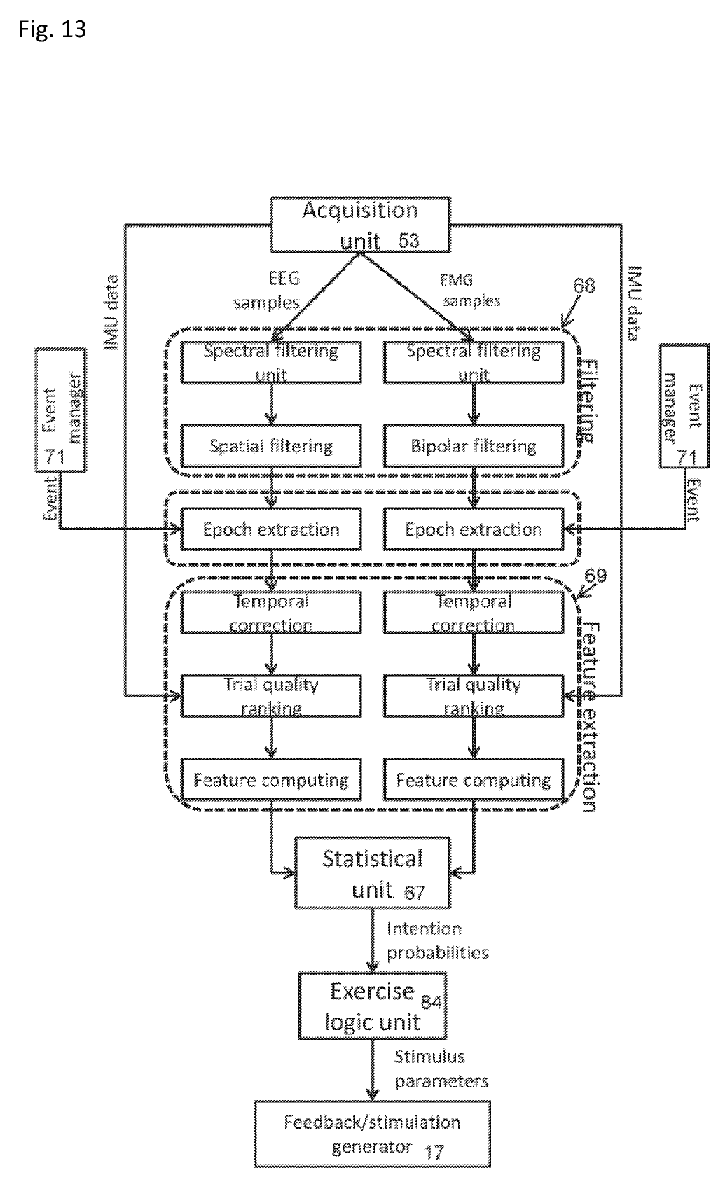

FIG. 13 illustrates various stages of the pre-processing (filtering 68, epoch extraction and feature extraction stages). EEG samples from all the electrodes are first spectrally filtered in various bands (e.g., 0.1-1 Hz, for slow cortical potentials, 8-12 Hz for alpha waves and Rolandic mu rhythms, 18-30 Hz for beta band and from 30-100 Hz for gamma band). Each of these spectral bands contains different aspects of neural oscillations at different locations. Following this stage, the signals undergo spatial filtering to improve signal-to-noise ratio additionally. The spatial filters include simple processes such as common average removal to spatial convolution with Gaussian window or Laplace windows. Following this stage, the incoming samples are segmented into temporal windows based on event markers arriving from event manager 71. These events correspond to the moment the user is given a stimulus or made a response.

These EEG segments are then fed to feature extraction unit 69, where temporal correction is first made. One simple example of temporal correction is removal of baseline or offset from the trial data from a selected spectral band data. The quality of these trials is assessed using statistical methods such as Outliers detection. Additionally, if there is a head movement registered through IMU sensor data, the trials are annotated as artefact trials. Finally, features are computed from each trial that well describe the underlying neural processing. These features are then fed to a statistical unit 67.

Similarly, the EMG electrode samples are first spectrally filtered, and applied a spatial filter. The movement information is obtained from the envelope or power of the EMG signals. Similar to EEG trials, EMG spectral data is segmented and passed to feature extraction unit 69. The output of EMG feature data is then sent to statistical unit 67.

The statistical unit 67 combines various physiological signals and motion data to interpret the intention of the user in performing a goal directed movement. This program unit includes mainly machine learning methods for detection, classification, and regression analysis in interpretation of the features. The outputs of this module are intention probabilities and related parameters which drive the logic of the exercise in the exercise logic unit 84. This exercise logic unit 84 generates stimulation parameters which are then sent to a feedback/stimulation generation unit of the stimulation system 17.

Throughout these stages, it is ensured to have minimal lag and more importantly least jitter.

Event Detection & Event Manager

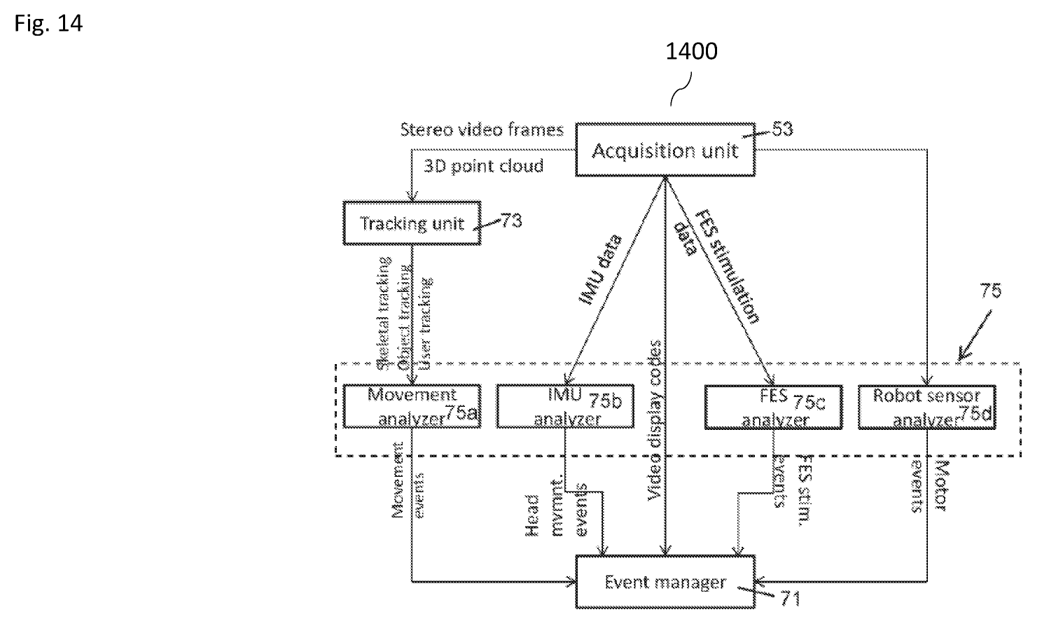

Events such as the moment at which the user is stimulated or presented an instruction in the VR display, the moment at which the user performed an action are necessary for the interpretation of the physiological data. FIG. 14 illustrates event detection. The events corresponding to movements and those of external objects or of a second person need to be detected. For this purpose, the data from camera system 30 (stereo cameras, and 3D point cloud from the depth sensor) are integrated in the tracking unit module 73 to produce various tracking information such as: (i) user's skeletal tracking data, (ii) object tracking data, and (iii) a second user tracking data. Based on the requirements of the behavioral analysis, these tracking data may be used for generating various events (e.g., the moment at which user lifts his hand to hold door knob).

IMU data provides head movement information. This data is analyzed to get events such as user moving head to look at the virtual door knob.

The video display codes correspond to the video content (e.g., display of virtual door knob, or any visual stimulation). These codes also represent visual events. Similarly, FES stimulation events, Robot movement and haptic feedback events are detected and transferred into event manager 71. Analyzer modules 75, including a movement analyzer 75a, an IMU analyzer 75b, an FES analyzer 75c, and a robot sensor analyzer 75d process the various sensor and stimulation signals for the event manager 71.

The event manager 71 then sends these events for tagging the physiological data, motion tracking data, etc. Additionally, these events also are sent to exercise logic unit for adapting the dynamics of exercise or challenges for the user.

Other Aspects of Control System

The control system interprets the incoming motion data, intention probabilities from the physiological data and activates exercise logic unit and generates stimulation/feedback parameters. The following blocks are main parts of the control system.
VR feedback: The motion data (skeletal tracking, object tracking, and user tracking data) is used for rendering 3D VR feedback on the head-mounted displays, in form of avatars and virtual objects.
Exercise logic unit 84: The exercise logic unit implements sequence of visual display frames including instructions and challenges (target task to perform, in various difficulty levels) to the user. The logic unit also reacts to the events of the event manager 71. Finally, this unit sends stimulation parameters to the stimulation unit.
Robot & FES stimulation generation unit: this unit generates inputs required to perform a targeted movement of the robotic system 41 and associated haptic feedback. Additionally, stimulation patterns (current intensity and electrode locations) for the FES module could be made synchronous and congruent to the user.

### Example 3: Brain Computer Interface and Motion Data Activated Neural Stimulation with Augmenter Reality Feedback

### Objective

A system that can provide precise neural stimulation in relation to the actions performed by a user in real world, resulting in reinforcement of neural patterns for intended behaviors.

### Description

Actions of the user and that of a second person and objects in the scene are captured with a camera system for behavioral analysis. Additionally, neural data is recorded with one of the modalities (EEG, ECOG, etc.) are synchronized with IMU data. The video captured from the camera system is interleaved with virtual objects to generate 3D augmented reality feedback and provided to the user though head-mounted display. Finally, appropriate neural stimulation parameters are generated in the control system and sent to the neural stimulation.

Delay and jitter between user's behavioral and physiological measures and neural stimulation should be optimized for effective reinforcement of the neural patterns.

The implementation of this example is similar to Example 2, except that the head mounted display (HMD) displays Augmented Reality content instead of Virtual Reality (see FIG. 2e). In other words, virtual objects are embedded in 3D seen captured using stereo camera and displayed on micro displays insuring first person perspective of the scene. Additionally, direct neural stimulation in implemented through such as deep brain stimulation and cortical stimulation, and non-invasive stimulations such as trans-cranial direct current stimulation (tDCS), trans-cranial alternating current stimulation (tACS), trans-cranial magnetic stimulation (TMS), and trans-cranial Ultrasonic stimulation. The system can advantageously use one or more than one stimulation modalities at time to optimize the effect. This system exploits the acquisition unit 53 described in the example 1.

### Example 4: Applications to Neural Marketing

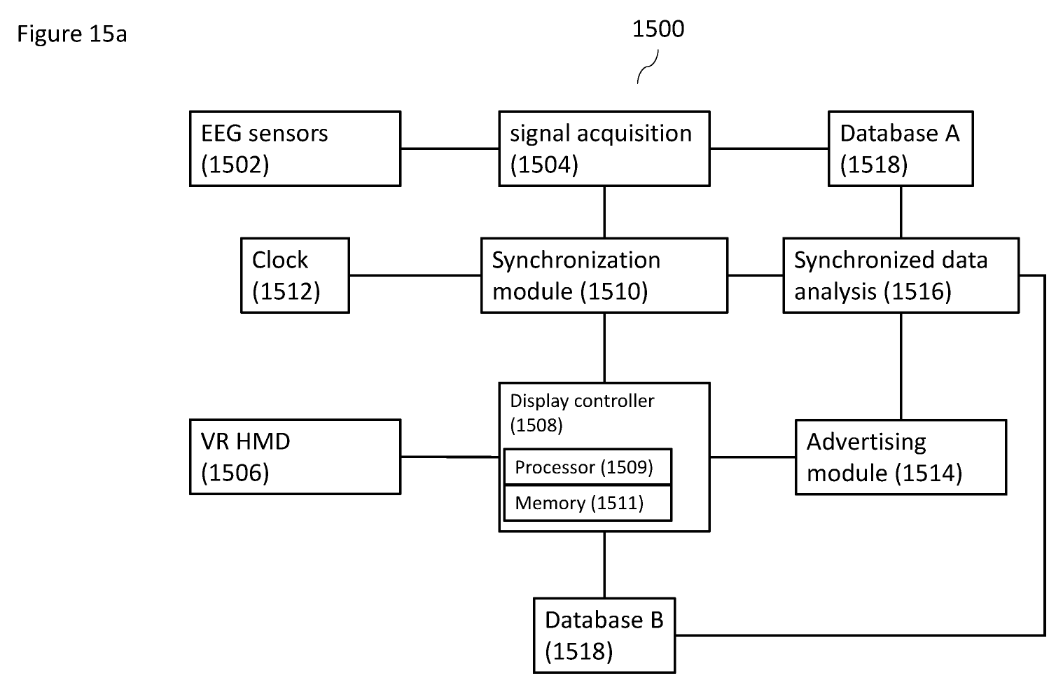

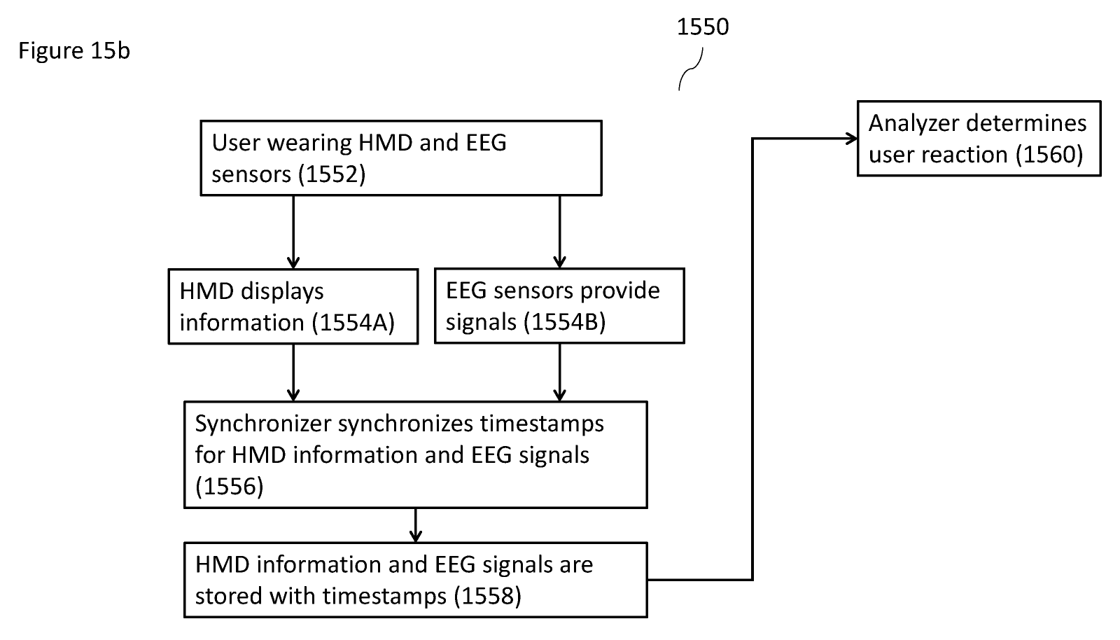

FIG. 15a shows an exemplary, non-limiting schematic block diagram for measuring an effect of visual stimuli on a reaction of an individual in a virtual reality environment. As noted above, a system 1400 is configured so that the sensor or stimulation data samples are attached with the time-stamp defined with the clock module. This means that complete synchronization between what was displayed and the exact reaction of the user is possible, as the data samples are synchronized to the display. As shown, a system 1500 features a plurality of EEG sensors 1502, which are preferably in contact with the scalp of the user as is known in the art, in order to collect EEG signals which are then fed to a signal acquisition module 1504. Signal acquisition module 1504 is optionally and preferably able to acquire signals from other types of physiological sensors as described herein, including EMG, EOG, ECG, inertial, body temperature, galvanic skin, respiration, pulse oximetry, and the like. EEG sensors 1502, signal acquisition module 1504, and other sensors can comprise a physiological parameter sensing system as described herein.

The user also preferably wears an HMD (head mounted display) 1506, which in this non-limiting example is for VR (virtual reality). A display controller 1508 feeds instructions and data to HMD 1506, to determine what the user views. Display controller 1508 and HMD 1506 may optionally be embodied in a single device or in a plurality of such devices.

Optionally display controller 1508 comprises a processor 1509 and a memory 1511. As used herein, a processor such as processor 1509 generally refers to a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory, such as memory 1511 in this non-limiting example. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

To provide synchronization between the information that the user views and the user's reaction to viewing such information, as noted above the acquired signals, such as EEG signals, are timestamped according to a timing that is synchronized with the same timestamp being applied to the flow of data to HMD 1506. A synchronization module 1510 provides such timestamp synchronization according to a clock 1512. Synchronization module 1510 communicates with signal acquisition module 1504 and display controller 1508, to provide timestamps for the data flowing through each of signal acquisition module 1504 and display controller 1508.

Data from signal acquisition module 1504 is optionally stored in a database A 1518 with the previously described timestamp, while data flowing through display controller 1508 is optionally stored in a database B 1518 with the previously described timestamp. A synchronized data analysis module 1516 optionally receives such synchronization information directly from synchronization module 1510 and may also receive data streams from one or both of signal acquisition module 1504 and display controller 1508.

Additionally, or alternatively, synchronized data analysis module 1516 may receive such data streams from each of databases A and B 1518. Preferably, synchronized data analysis module 1516 is in communication with an advertising module 1514, to determine which advertisements correspond to the data input to display controller 1508. An advertisement may be defined according to one or more images, or one or more sounds, a story comprising a plurality of such images and sounds, and so forth. The advertisement may also be defined according to a plurality of parameters that relate to a specific product or service being sold, a category of such products and services, and so forth. The image may be a logo or other icon.

Optionally, advertising module 1514 may be used to provide a game for display, preferably for a game with advertisements and/or to test the pace of a game and/or a new game character or game level.

Optionally synchronized data analysis module 1516 is able to determine the reaction of the user to information displayed by HMD 1506 according to an analysis of the EEG signals, as described for example in US Patent Publ. 20110282231, hereby incorporated by reference as if fully set forth herein.

Optionally the EEG sensors and HMD may be implemented according to any of the above Figures.

FIG. 15b shows an exemplary, non-limiting process for determining an effect of an advertisement on a user in a virtual reality environment. As shown in a process 1550, the user wears a VR HMD and also EEG sensors in 1552. Simultaneously or near-simultaneously, information is displayed in the HMD in 1554A while EEG signals are collected in 1554B. The information may include images and/or sounds, for example in the form of video data.

Next, in 1556, the information displayed in the HMD and the EEG signals are synchronized by a synchronizer with a timestamp. The synchronizer preferably operates according to a clock as previously described. HMD information and EEG signals are optionally stored with timestamps in 1558. Preferably, the reaction of the user to the information being displayed on the HMD is determined according to the EEG signals, such as for example the reaction of the user to a product (virtually displayed) or to an advertisement, in 1560.

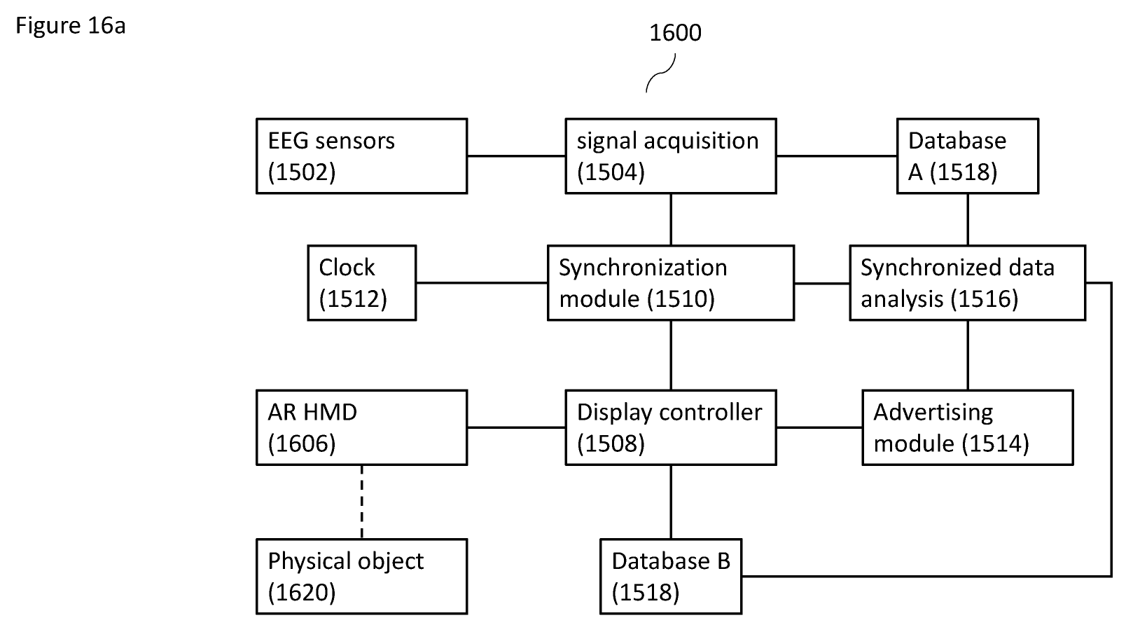

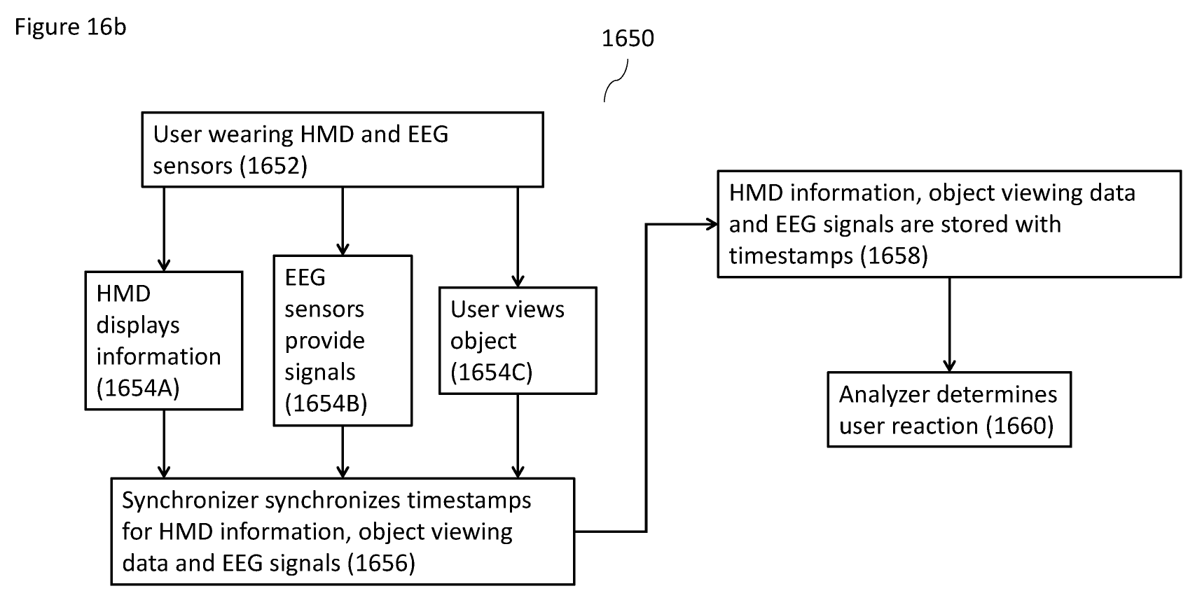

FIG. 16a shows an exemplary, non-limiting schematic block diagram for measuring an effect of visual stimuli on a reaction of an individual in an augmented reality environment. A system 1600 preferably operates similarly to the system of FIG. 15a, except that the HMD is now an AR (augmented reality) HMD 1606. Components with the same number as FIG. 15a have the same or similar function.

In addition, preferably a physical object 1620 is at least visible to the user through AR HMD 1606, as indicated by the dotted line. Optionally the user is able to handle physical object 1620. Also, preferably, video data regarding how and when the user views physical object 1620 is recorded, for example by HMD 1606, or alternatively or additionally by another video camera (not shown). This information preferably also receives timestamps by synchronization module 1510 and is preferably stored with the timestamps in database B 1518. Preferably, synchronized data analysis 1516 is able to correlate how and when the user views physical object 1620 with the EEG signals, for example to determine the user reaction to the object and/or to information being displayed by HMD 1606.

FIG. 16b shows an exemplary, non-limiting process for determining an effect of an advertisement on a user in an augmented reality environment. As shown in a process 1650, the user wears an AR HMD and also EEG sensors in 1652 and is preferably able to at least view a physical object. More preferably the user is able to handle the physical object. Simultaneously or near-simultaneously, the user preferably at least views the object and more preferably handles the object in 1654C, while information is displayed in the HMD in 1654A and EEG signals are collected in 1654B. The information may include images and/or sounds, for example in the form of video data. Also, preferably, video data about the user at least viewing the object and more preferably handling the object is collected in 1654C.

Next, in 1656, the video data of the user at least viewing (if not actually handling) the object, information displayed in the HMD and the EEG signals are synchronized by a synchronizer with a timestamp. The synchronizer preferably operates according to a clock as previously described. HMD information, user viewing information and EEG signals are optionally stored with timestamps in 1658. Preferably, the reaction of the user to the object, the information being displayed on the HMD is determined according to the EEG signals, such as for example the reaction of the user to a product (virtually displayed) or to an advertisement, in 1660.

Any and all references to publications or other documents, including but not limited to, patents, patent applications, articles, webpages, books, etc., presented in the present application, are herein incorporated by reference in their entirety.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices, including any and all elements corresponding to systems, methods, and apparatuses/device for tracking a body or portions thereof. In other words, elements from one or another disclosed embodiment may be interchangeable with elements from other disclosed embodiments. In addition, one or more features/elements of disclosed embodiments may be removed and still result in patentable subject matter (and thus, resulting in yet more embodiments of the subject disclosure). Correspondingly, some embodiments of the present disclosure may be patentably distinct from one and/or another reference by specifically lacking one or more elements/features. In other words, claims to certain embodiments may contain negative limitation to specifically exclude one or more elements/features resulting in embodiments which are patentably distinct from the prior art which include such features/elements.

1. A physiological parameter measurement and motion tracking system comprising:
a VR or AR display system to display information to a user;
a physiological parameter sensing system comprising one or more sensing means configured to sense electrical activity in a brain of a user and to generate brain electrical activity information;
a synchronizer to provide timestamps of said information displayed to the user and said brain electrical activity information, said synchronizer comprising a clock for determining said timestamps; and
an analyzer arranged to receive the brain electrical activity information and the displayed information with said timestamps, to determine a reaction of the user to the displayed information according to the brain electrical activity information.

2. The system of claim 1, wherein said display information comprises a plurality of images and/or sounds.

3. The system of claim 2, wherein said display information comprises a video stream.

4. The system of 3, further comprising an advertising module for providing the display information to the display system as advertising information, wherein said analyzer determines a reaction of the user to said advertising information.

5. The system of claim 4, wherein said display system comprises an AR HMD through which a physical object is viewable, and which includes a video camera for recording when and how the user views the physical object, said synchronizer is configured to apply a timestamp to video data for determining when and how the user views the physical object, and said analyzer determines said reaction of the user also according to said timestamp of video data of when and how the user views the physical object.

6. A physiological parameter measurement and motion tracking system comprising:
a VR or AR display system to display information to a user;
a physiological parameter sensing system comprising (i) one or more sensing means configured to sense electrical activity in a brain of a user and to generate brain electrical activity information and (ii) one or more of an EMG sensor, EOG sensor, ECG sensor, body temperature sensor, galvanic skin sensor, and respiration sensor; and (iii) a signal acquisition module configured to acquire a signal from at least one of the EMG sensor, EOG sensor, ECG sensor, body temperature sensor, galvanic skin sensor, and respiration sensor;
a synchronizer to provide timestamps of said information displayed to the user, said brain electrical activity information, and said signal from the at least one of the EMG sensor, EOG sensor, ECG sensor, body temperature sensor, galvanic skin sensor, and respiration sensor, said synchronizer comprising a clock for determining said timestamps; and
an analyzer arranged to receive said brain electrical activity information, said signal from the at least one of the EMG sensor, EOG sensor, ECG sensor, body temperature sensor, galvanic skin sensor, and respiration sensor, and the displayed information with said timestamps, to determine a reaction of the user to the displayed information according to the brain electrical activity information.

7. A method for physiological parameter measurement, comprising:
receiving display information configured for an HMD;
receiving an EEG sensor signal;
synchronizing, using a synchronizer module, the display information and the EEG sensor signal to generate synchronized data;
storing, the synchronized data; and
analyzing the synchronized data to determine a user reaction;
wherein the synchronizing includes associating a timestamp with the display information and the EEG signal, the timestamp generated from a single clock module.

8. The method of claim 7, further comprising:
receiving a signal from at least one of an EMG sensor, EOG sensor, ECG sensor, body temperature sensor, galvanic skin sensor, and respiration sensor; and
wherein the synchronizing further includes associating the timestamp with the signal from the at least one of the EMG sensor, EOG sensor, ECG sensor, body temperature sensor, galvanic skin sensor, and respiration sensor.

9. The method of claim 7, further comprising:
generating the display information using an advertising module.
